(19) United States
(12) Patent Application Publication
(10) Pub. No.: US 2025/0017933 A1
(43) Pub. Date: Jan. 16, 2025

(54) CANCER TREATMENT USING LSD1 INHIBITORS AND PLK1 INHIBITORS

(71) Applicant: Cardiff Oncology, Inc., San Diego, CA (US)

(72) Inventors: Maya Ridinger, San Diego, CA (US); Anju Karki, San Diego, CA (US)

(21) Appl. No.: 18/710,027

(22) PCT Filed: Nov. 16, 2022
(86) PCT No.: PCT/US2022/079939

§ 371 Date: May 14, 2024

Related U.S. Application Data
(60) Provisional application No. 63/280,565, filed on Nov. 17, 2021.
(60) Provisional application No. 63/310,431, filed on Feb. 15, 2022.

(51) Int. Cl. A61K 31/517; A61K 31/496; A61K 45/06; A61P 35/00; C12Q 1/6886

(52) U.S. Cl. CPC A61K 31/517; A61K 31/496; A61K 45/06; A61P 35/00; C12Q 1/6886; C12Q 2600/106

(57) ABSTRACT

Provided include methods, compositions and kits for treating cancer in a subject. The method can comprise administrating an LSD1 inhibitor and a PLK1 inhibitor (for example, onvansertib) to the subject in a manner sufficient to inhibit or reduce progression of the cancer.

### RELATED APPLICATIONS

The present application is a U.S. national phase application under 35 U.S.C. § 371 of International Application No. PCT/US2022/079939, filed on Nov. 16, 2022 and published as WO 2023/091932 A1 on May 25, 2023, which claims priority under 35 U.S.C. § 119(e) to U.S. Provisional Application No. 63/280,565 filed Nov. 17, 2021 and U.S. Provisional Application No. 63/310,431 filed Feb. 15, 2022; the content of each of which is hereby expressly incorporated by reference in its entirety.

### BACKGROUND

### Field

The present application generally relates to treatment for cancer. More specifically, combination therapies for treating cancer using lysine-specific histone demethylase 1 (LSD1) inhibitors in combination with polo-like kinase 1 (PLK1) inhibitors are provided.

### Description of the Related Art

Polo-like kinase 1 (PLK1) is a well characterized member of the 5 members of the family of serine/threonine protein kinases and strongly promotes the progression of cells through mitosis. PLK1 performs several important functions throughout mitotic (M) phase of the cell cycle, including the regulation of centrosome maturation and spindle assembly, the removal of cohesins from chromosome arms, the inactivation of anaphase-promoting complex/cyclosome (APC/C) inhibitors, and the regulation of mitotic exit and cytokinesis. PLK1 plays a key role in centrosome functions and the assembly of bipolar spindles. PLK1 also acts as a negative regulator of p53 family members leading to ubiquitination and subsequent degradation of p53/TP53, inhibition of the p73/TP73 mediated pro-apoptotic functions and phosphorylation/degradation of bora, a cofactor of Aurora kinase A. During the various stages of mitosis PLK1 localizes to the centrosomes, kinetochores and central spindle. PLK1 is a master regulator of mitosis and aberrantly overexpressed in a variety of human cancers including AML and is correlated with cellular proliferation and poor prognosis.

LSD1 inhibitors are inhibitors of lysine-specific histone demethylase 1 (LSD1). LSD1 is a flavin adenine dinucleotide (FAD)-dependent amine oxidase, was the first identified demethylase (eraser) for lysine methylation of histones and non-histone proteins. LSD1 is also known as lysine (K)-specific demethylase 1A (KDM1A) is a protein in humans that is encoded by the KDM1A gene. LSD1 specifically removes methyl groups via a redox process of mono- or di-methylated histone H3 lysine 4 (H3K4) and H3 lysine 9 (H3K9). LSD1 in the CoREST-HDAC containing repressor complexes, functions as a corepressor by mediating demethylation of H3K4me. In addition, LSD1 is recruited to the promoter regions of androgen receptor (AR) target genes and demethylates H3K9me, co-activating AR-dependent transcription. Misregulated expression of LSD1 has been reported in several cancer types. LSD1 gene deletion was reported in pancreatic ductal adenocarcinoma, while its gene amplification was found in neuroendocrine prostate cancer and sarcoma.

There is a need to develop effective treatment for cancer patients, including the patients with LSD1-altered tumors and patients resistant to LSD1 inhibitor treatment.

### SUMMARY

Provided include methods, compositions and kits for treating cancer. Some embodiments provide a method of treating cancer, where the method comprises: administrating a lysine-specific histone demethylase 1 (LSD1) inhibitor and a polo-like kinase 1 (PLK1) inhibitor to a subject with cancer, thereby inhibiting or reducing progression of the cancer in the subject. Some embodiments provide a method of sensitizing cancer cells to a LSD1 inhibitor, the method comprising: contacting cancer cells with a composition comprising a PLK1 inhibitor, thereby sensitizing the cancer cells to the LSD1 inhibitor. Also provided are some embodiments related to kits, where the kit comprises a PLK1 inhibitor; and a manual providing instructions for co-administrating the PLK1 inhibitor with a LSD1 inhibitor to a subject in need thereof for treating cancer.

The cancer can be, for example, head and neck cancer, lung cancer, intrahepatic cholangiocarcinoma (iCCA), gastric cancer, urothelial cancer, endometrial cancer, cervical cancer, rhabdomyosarcoma, cholangiocarcinoma, glioblastoma, low-grade glioma, ovarian cancer, prostate adenocarcinoma, thyroid carcinoma, endometrial cancer, gallbladder cancer, breast cancer, pancreatic ductal adenocarcinoma, prostate cancer, sarcoma, or a combination thereof. In some embodiments, the prostate cancer is neuroendocrine prostate cancer. In some embodiments, the subject has overexpression of LSD1 gene or amplification of LSD1 gene. In some embodiments, the PLK1 inhibitor is onvansertib.

### BRIEF DESCRIPTION OF THE DRAWINGS

FIGS. 1A-1E show representative synergy scores in small cell lung cancer (SCLC) cell lines SHP77, DMS53, DMS 114, H1417, and H69 after treatment with various doses of onvansertib and ORY-1001 for 6 days. A positive score indicate synergy between onvansertib and ORY-1001.

FIGS. 2A-2E show representative cell viability results in SCLC cell lines SHP77, DMS53, DMS114, H1417, and H69 after treatment with various doses of ORY-1001 in the absence or presence of onvansertib. Expected viability results calculated using the Bliss independence model of drug additivity for each cell line are also shown.

FIGS. 3A-E are plots showing changes in tumor volume in a LTL331R78 PDX model treated with vehicle, onvansertib, IMG-7289, or combination of onvansertib and IMG-7289.

FIGS. 4A-C are plots showing changes in tumor volume in an EF1 PDX model treated with vehicle, onvansertib, IMG-7289, or combination of onvansertib and IMG-7289.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein and made part of the disclosure herein.

All patents, published patent applications, other publications, and sequences from GenBank, and other databases referred to herein are incorporated by reference in their entirety with respect to the related technology.

### Definitions

Unless defined otherwise, technical and scientific terms used herein have the same meaning as commonly understood by one of ordinary skill in the art to which the present disclosure belongs. See, e.g. Singleton et al., Dictionary of Microbiology and Molecular Biology 2nd ed., J. Wiley & Sons (New York, NY 1994); Sambrook et al., Molecular Cloning, A Laboratory Manual, Cold Spring Harbor Press (Cold Spring Harbor, NY 1989). For purposes of the present disclosure, the following terms are defined below.

As used herein, a “subject” refers to an animal that is the object of treatment, observation or experiment. “Animals” include cold- and warm-blooded vertebrates and invertebrates such as fish, shellfish, reptiles and, in particular, mammals. “Mammal” includes, without limitation, mice; rats; rabbits; guinea pigs; dogs; cats; sheep; goats; cows; horses; primates, such as monkeys, chimpanzees, and apes, and, in particular, humans.

As used herein, a “patient” refers to a subject that is being treated by a medical professional, such as a Medical Doctor (i.e., Doctor of Allopathic medicine or Doctor of Osteopathic medicine) or a Doctor of Veterinary Medicine, to attempt to cure, or at least ameliorate the effects of, a particular disease or disorder or to prevent the disease or disorder from occurring in the first place. In some embodiments, the patient is a human or an animal. In some embodiments, the patient is a mammal.

As used herein, “administration” or “administering” refers to a method of giving a dosage of a pharmaceutically active ingredient to a vertebrate.

As used herein, a “dosage” refers to the combined amount of the active ingredients (e.g., cyclosporine analogues, including CRV431).

As used herein, a “unit dosage” refers to an amount of therapeutic agent administered to a patient in a single dose.

As used herein, the term “daily dose” or “daily dosage” refers to a total amount of a pharmaceutical composition or a therapeutic agent that is to be taken within 24 hours.

As used herein, the term “delivery” refers to approaches, formulations, technologies, and systems for transporting a pharmaceutical composition or a therapeutic agent into the body of a patient as needed to safely achieve its desired therapeutic effect. In some embodiments, an effective amount of the composition or agent is formulated for delivery into the blood stream of a patient.

As used herein, the term “formulated” or “formulation” refers to the process in which different chemical substances, including one or more pharmaceutically active ingredients, are combined to produce a dosage form. In some embodiments, two or more pharmaceutically active ingredients can be co-formulated into a single dosage form or combined dosage unit, or formulated separately and subsequently combined into a combined dosage unit. A sustained release formulation is a formulation which is designed to slowly release a therapeutic agent in the body over an extended period of time, whereas an immediate release formulation is a formulation which is designed to quickly release a therapeutic agent in the body over a shortened period of time.

As used herein, the term “pharmaceutically acceptable” indicates that the indicated material does not have properties that would cause a reasonably prudent medical practitioner to avoid administration of the material to a patient, taking into consideration the disease or conditions to be treated and the respective route of administration. For example, it is commonly required that such a material be essentially sterile.

As used herein, the term “pharmaceutically acceptable carrier” refers to pharmaceutically acceptable materials, compositions or vehicles, such as a liquid or solid filler, diluent, excipient, solvent or encapsulating material, involved in carrying or transporting any supplement or composition, or component thereof, from one organ, or portion of the body, to another organ, or portion of the body, or to deliver an agent to a diseased tissue or a tissue adjacent to the diseased tissue. Carriers or excipients can be used to produce compositions. The carriers or excipients can be chosen to facilitate administration of a drug or pro-drug. Examples of carriers include calcium carbonate, calcium phosphate, various sugars such as lactose, glucose, or sucrose, or types of starch, cellulose derivatives, gelatin, vegetable oils, polyethylene glycols and physiologically compatible solvents. Examples of physiologically compatible solvents include sterile solutions of water for injection (WFI), saline solution, and dextrose.

As used herein, the term “pharmaceutically acceptable salt” refers to any acid or base addition salt whose counter-ions are non-toxic to the patient in pharmaceutical doses of the salts. A host of pharmaceutically acceptable salts are well known in the pharmaceutical field. If pharmaceutically acceptable salts of the compounds of this disclosure are utilized in these compositions, those salts are preferably derived from inorganic or organic acids and bases. Included among such acid salts are the following: acetate, adipate, alginate, aspartate, benzoate, benzene sulfonate, bisulfate, butyrate, citrate, camphorate, camphor sulfonate, cyclopentanepropionate, digluconate, dodecylsulfate, ethanesulfonate, fumarate, lucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, pamoate, pectinate, persulfate, 3-phenyl-propionate, picrate, pivalate, propionate, succinate, tartrate, thiocyanate, tosylate, undecanoate, hydrohalides (e.g., hydrochlorides and hydrobromides), sulphates, phosphates, nitrates, sulphamates, malonates, salicylates, methylene-bis-b-hydroxynaphthoates, gentisates, isethionates, di-p-toluoyltartrates, ethanesulphonates, cyclohexylsulphamates, quinates, and the like. Pharmaceutically acceptable base addition salts include, without limitation, those derived from alkali or alkaline earth metal bases or conventional organic bases, such as triethylamine, pyridine, piperidine, morpholine, N-methylmorpholine, ammonium salts, alkali metal salts, such as sodium and potassium salts, alkaline earth metal salts, such as calcium and magnesium salts, salts with organic bases, such as dicyclohexylamine salts, N-methyl-D-glucamine, and salts with amino acids such as arginine, lysine, and so forth.

As used herein, the term “hydrate” refers to a complex formed by combination of water molecules with molecules or ions of the solute. As used herein, the term “solvate” refers to a complex formed by combination of solvent molecules with molecules or ions of the solute. The solvent can be an organic compound, an inorganic compound, or a mixture of both. Solvate is meant to include hydrate, hemi-hydrate, channel hydrate etc. Some examples of solvents include, but are not limited to, methanol, N,N-dimethylformamide, tetrahydrofuran, dimethylsulfoxide, and water.

As used herein, “therapeutically effective amount” or “pharmaceutically effective amount” refers to an amount of therapeutic agent, which has a therapeutic effect. The dosages of a pharmaceutically active ingredient which are useful in treatment when administered alone or in combination with one or more additional therapeutic agents are therapeutically effective amounts. Thus, as used herein, a therapeutically effective amount refers to an amount of therapeutic agent which produces the desired therapeutic effect as judged by clinical trial results and/or model animal studies. The therapeutically effective amount will vary depending on the compound, the disease, disorder or condition and its severity and the age, weight, etc., of the mammal to be treated. The dosage can be conveniently administered, e.g., in divided doses up to four times a day or in sustained-release form.

As used herein, the term “treat,” “treatment,” or “treating,” refers to administering a therapeutic agent or pharmaceutical composition to a subject for prophylactic and/or therapeutic purposes. The term “prophylactic treatment” refers to treating a subject who does not yet exhibit symptoms of a disease or condition, but who is susceptible to, or otherwise at risk of, a particular disease or condition, whereby the treatment reduces the likelihood that the patient will develop the disease or condition. The term “therapeutic treatment” refers to administering treatment to a subject already suffering from a disease or condition. As used herein, a “therapeutic effect” relieves, to some extent, one or more of the symptoms of a disease or disorder. For example, a therapeutic effect may be observed by a reduction of the subjective discomfort that is communicated by a subject (e.g., reduced discomfort noted in self-administered patient questionnaire).

As used herein, the term “prophylaxis,” “prevent,” “preventing,” “prevention,” and grammatical variations thereof as used herein refers the preventive treatment of a subclinical disease-state in a subject, e.g., a mammal (including a human), for reducing the probability of the occurrence of a clinical disease-state. The method can partially or completely delay or preclude the onset or recurrence of a disorder or condition and/or one or more of its attendant symptoms or barring a subject from acquiring or reacquiring a disorder or condition or reducing a subject's risk of acquiring or requiring a disorder or condition or one or more of its attendant symptoms. The subject is selected for preventative therapy based on factors that are known to increase risk of suffering a clinical disease state compared to the general population. “Prophylaxis” therapies can be divided into (a) primary prevention and (b) secondary prevention. Primary prevention is defined as treatment in a subject that has not yet presented with a clinical disease state, whereas secondary prevention is defined as preventing a second occurrence of the same or similar clinical disease state.

As used herein, each of the terms “partial response” and “partial remission” refers to the amelioration of a cancerous state, as measured by, for example, tumor size and/or cancer marker levels, in response to a treatment. In some embodiments, a “partial response” means that a tumor or tumor-indicating blood marker has decreased in size or level by about 50% in response to a treatment. The treatment can be any treatment directed against cancer, including but not limited to, chemotherapy, radiation therapy, hormone therapy, surgery, cell or bone marrow transplantation, and immunotherapy. The size of a tumor can be detected by clinical or by radiological means. Tumor-indicating markers can be detected by means well known to those of skill, e.g., ELISA or other antibody-based tests.

As used herein, each of the terms “complete response” or “complete remission” means that a cancerous state, as measured by, for example, tumor size and/or cancer marker levels, has disappeared following a treatment, including but are not limited to, chemotherapy, radiation therapy, hormone therapy, surgery, cell or bone marrow transplantation, and immunotherapy. The presence of a tumor can be detected by clinical or by radiological means. Tumor-indicating markers can be detected by means well known to those of skill, e.g., ELISA or other antibody-based tests. A “complete response” does not necessarily indicate that the cancer has been cured, however, as a complete response can be followed by a relapse.

### Cancer

Methods, compositions and kits disclosed herein can be used for treating cancer. In some embodiments, a method for treating cancer comprises administrating an LSD1 inhibitor, or a pharmaceutically acceptable salt, solvate, stereoisomer thereof, and a Polo-like kinase 1 (PLK1) inhibitor (e.g., onvansertib), or a pharmaceutically acceptable salt, solvate, stereoisomer thereof, to a subject (e.g., a patient) in need thereof.

The methods, compositions and kits disclosed herein can be used to various types of cancer, including but are not limited to, melanoma (e.g., metastatic malignant melanoma), renal cancer (e.g., clear cell carcinoma), prostate cancer (e.g., hormone refractory prostate adenocarcinoma), pancreatic adenocarcinoma, breast cancer, colon cancer, lung cancer (e.g., non-small cell lung cancer (NSCLC) and small-cell lung cancer (SCLC)), esophageal cancer, squamous cell carcinoma of the head and neck, liver cancer, ovarian cancer, cervical cancer, thyroid cancer, glioblastoma, glioma, leukemia, lymphoma, and other neoplastic malignancies. Additionally, the disease or condition provided herein includes refractory or recurrent malignancies whose growth may be inhibited using the methods and compositions disclosed herein. In some embodiments, the cancer is carcinoma, squamous carcinoma, adenocarcinoma, sarcomata, endometrial cancer, breast cancer, ovarian cancer, cervical cancer, fallopian tube cancer, primary peritoneal cancer, colon cancer, colorectal cancer, squamous cell carcinoma of the anogenital region, melanoma, renal cell carcinoma, lung cancer, non-small cell lung cancer, squamous cell carcinoma of the lung, stomach cancer, bladder cancer, gall bladder cancer, liver cancer, thyroid cancer, laryngeal cancer, salivary gland cancer, esophageal cancer, head and neck cancer, glioblastoma, glioma, squamous cell carcinoma of the head and neck, prostate cancer, pancreatic cancer, mesothelioma, sarcoma, hematological cancer, leukemia, lymphoma, neuroma, or a combination thereof. In some embodiments, the cancer is carcinoma, squamous carcinoma (e.g., cancer of cervical canal, eyelid, tunica conjunctiva, vagina, lung, oral cavity, skin, urinary bladder, tongue, larynx, and gullet), and adenocarcinoma (for example, cancer of prostate, small intestine, endometrium, cervical canal, large intestine, lung, pancreas, gullet, rectum, uterus, stomach, mammary gland, and ovary). In some embodiments, the cancer is sarcomata (e.g., myogenic sarcoma), leukosis, neuroma, melanoma, and lymphoma. In some embodiments, the cancer is bone cancer, breast cancer, brain tumor, central nervous system tumor, colorectal cancer, connective tissue cancer, endometrial cancer, esophageal cancer, gastric cancer, head and neck cancer, kidney cancer, leukemia, liver cancer, lung cancer, lymphoma, myeloma, ovarian cancer, pancreatic cancer, prostate cancer, skin cancer, soft tissue sarcoma, thyroid cancer, or bladder cancer.

The cancer can be a solid tumor, a liquid tumor, or a combination thereof. In some embodiments, the cancer is a solid tumor, including but are not limited to, melanoma, renal cell carcinoma, lung cancer, bladder cancer, breast cancer, cervical cancer, colon cancer, gall bladder cancer, laryngeal cancer, liver cancer, thyroid cancer, stomach cancer, salivary gland cancer, prostate cancer, pancreatic cancer, Merkel cell carcinoma, brain and central nervous system cancers, and any combination thereof. In some embodiments, the cancer is a liquid tumor. In some embodiments, the cancer is a hematological cancer. Non-limiting examples of hematological cancer include Diffuse large B cell lymphoma (“DLBCL”), Hodgkin's lymphoma (“HL”), Non-Hodgkin's lymphoma (“NHL”), Follicular lymphoma (“FL”), acute myeloid leukemia (“AML”), and multiple myeloma (“MM”).

The cancer can be LSD1 altered cancer which comprises one or more LSD1 alterations and/or LSD1 aberrant activation such as copy number alteration (CNA), single-nucleotide variation (SNV), and gene rearrangement or fusions. Non-limiting exemplary cancer with LSD1 alterations include cancer with LSD1 gene amplification, LSD1 gene deletion, LSD1 overexpression, high LSD1 expression, and combination thereof.

In some embodiments, the cancer can be LSD1-amplified cancer in which LSD1 gene is amplified, for example, as a result of gene duplication or aberrant gene transcriptional control. For example, the cancer with LSD1 amplification can be prostate cancer with LSD1 amplification (e.g., neuroendocrine prostate cancer). In some embodiments, the prostate cancer is resistant to LSD1 inhibitor treatment.

In some embodiments, the cancer is a lung cancer. The lung cancer can be, for example, NSCLC and SCLC (also known as oat cell lung cancer). NSCLC can comprise subcategories such as adenocarcinoma, squamous cell carcinoma (SqCC), large cell carcinoma, and other cancer types including adenosquamous carcinoma and sarcomatoid carcinoma. In some embodiments, the lung cancer is NSCLC, SqCC, NSCLC adenocarcinoma, NSCLC large cell carcinoma, and/or SCLC. The lung cancer can be, in some embodiments, pulmonary metastases or pulmonary neuroendocrine tumor (including but not limited to large cell neuroendocrine carcinoma, typical carcinoid tumor, and atypical carcinoid tumor).

### LSD1 Inhibitors and PLK Inhibitors

Methods, compositions and kits disclosed herein can be used for treating cancer, for example lung cancer, cervical cancer, urothelial cancer, gastric cancer intrahepatic cholangiocarcinoma, endometrial cancer, rhabdomyosarcoma, cholangiocarcinoma, ovarian cancer, breast cancer, prostate cancer, colorectal cancer, pancreatic cancer, or a combination thereof. In some embodiments, a method for treating cancer comprises administrating a LSD1 inhibitor, or a pharmaceutically acceptable salt, solvate, stereoisomer thereof, and a PLK1 inhibitor (e.g., onvansertib), or a pharmaceutically acceptable salt, solvate, stereoisomer thereof, to a subject (e.g., a patient) in need thereof. The method can comprise administering a pharmaceutically effective amount of the LSD1 inhibitor and a pharmaceutically effective amount of the PLK1 inhibitor (e.g., onvansertib).

### LSD1 Inhibitors

Lysine-specific demethylase (LSD1), also known as lysine (K)-specific demethylase 1A (LSD1), is a protein in humans that in encoded by the KDM1A gene and specifically demethylates mono- or dimethylated histone H3 lysine 4 (H3K4) and H3 lysine 9 (H3K9) via a redox process. LSD1 functions in the regulation of gene expression as a transcriptional repressor or activator, and plays a pivotal role in various physiological processes, including development, differentiation, inflammation, thermogenesis, neuronal and cerebral physiology, and the maintenance of stemness in stem cells. LSD1 also participates in pathological processes, including cancer as the most representative disease. LSD1 promotes oncogenesis by facilitating the survival of cancer cells and by generating a pro-cancer microenvironment.

Lysine methyltransferases and demethylases catalyze the process of N-methylation and N-demethylation of histone lysines, respectively. Based on the catalytic mechanisms, the demethylases are divided into two subgroups: the flavin adenine dinucleotide (FAD)-dependent LSD1 and LSD2 and JMJD family containing JmjC domain. LSD1 specifically demethylates histone lysine residues H3K4me1/2 and H3K9 me1/2.

Methylated histone marks on K3K4 and H3K9 are generally coupled with transcriptional activation and repression, respectively. As part of corepressor complexes (e.g., CoREST), LSD1 has been reported to demethylate H3K4 and repress transcription, whereas LSD1, in nuclear hormone receptor complex (e.g., androgen receptor), may demethylate H3K9 to activate gene expression. This suggests the substrate specificity of LSD1 can be determined by associated factors, thereby regulating alternative gene expressions in a context dependent manner. In addition to histone proteins, LSD1 can demethylate non-histone proteins (including but not limited to p53, E2F, STAT3, Tat, and myosin phosphatase target subunit 1 (MYPT1)), which indicates additional oncogenic roles of LSD1 beyond in regulating chromatin remodeling. LSD1 also associates with other epigenetic regulators, such as DNA methyltransferase 1 (DNMT1) and histone deacetylases (HDACs) complexes. These associations augment the activities of DNMT or HDACs. LSD1 inhibitors may therefore potentiate the effects of HDAC or DNMT inhibitors. Indeed, preclinical studies have shown such potential already. LSD1 has been reported to contribute to a variety of biological processes, including cell proliferation, epithelial-mesenchymal transition (EMT), and stem cell biology (both embryonic stem cells and cancer stem cells) or self-renewal and cellular transformation of somatic cells.

LSD1 has three structural domains that regulate its enzymatic activity and binding to several proteins. From the N-terminus this enzyme consists of a SWIRM domain (named after the Swi3p, Rsc8p, and Moira proteins in which it was first identified), a Tower domain and a C-terminal FAD-dependent AO domain that surrounds the Tower domain and consists of two different lobes. A disordered area of 170 residues, which can be post-translationally modified and promotes protein-protein interactions, characterizes the N-terminal extremity. The SWIRM domain follows this region and is shaped as a small alpha-helix that lacks the DNA-binding ability typical of other SWIRM domains but maintains its protein-protein interaction ability. The AO domain is the catalytic region of LSD1 and consists of two lobes, the FAD-binding site and the substrate recognition site, shaped in a more open conformation than any other FAD-dependent monoamine oxidase. In the tertiary structure of LSD1, the second lobe of the AO domain is in close proximity to the SWIRM domain, thus forming a hydrophobic groove that allows LSD1 to accommodate a large portion of histone H3 tail, sense epigenetic marks, and modify chromatin accessibility thought its demethylating activity. Finally, the Tower domain consists of two antiparallel helices that in the tertiary structure of LSD1 protrude from the AO domain and act as a platform for binding to RCOR1, a member of the CoREST transcriptional repressor complex. LSD1 exerts dual functions by acting as a transcription co-repressor or a co-activator through H3K4 or H3K9 demethylation, respectively.

Elevated levels of LSD1 has been found in diverse cancers and shows close relationship with many cellular effects such as epithelial-mesenchymal transition (EMT), cell proliferation and differentiation, stem cell biology, and malignant transformation. LSD1 inactivation also enhances anti-tumor immunity and inhibits checkpoint. LSD1 dysfunction is also associated with the development of acute lymphoblastic leukemia (ALL) and acute myeloid leukemia (AML). Preclinical studies showed that LSD1 inhibition can suppress tumor growth of lung adenocarcinoma independent on driver mutations. Expression profiling reveals that LSD1 inhibition mainly affects replication machinery and cell cycle, and interrupts downstream signaling of EGFR (epidermal growth factor receptor). Pharmacological inhibition of LSD1 leads to inhibition of proliferation, differentiation, invasion, and migration in vitro and in vivo.

Cancer stem cells or cancer initiating cells have some pluripotent stem cell properties that contribute to the heterogeneity of cancer cells, which can render cancer cells more resistant to conventional therapies, such as chemotherapy or radiotherapy, and then develop recurrence after treatment. LSD1 was reported to maintain an undifferentiated tumor initiating or cancer stem cell phenotype in a spectrum of cancers. For example, analysis of AML cells including gene expression arrays and chromatin immunoprecipitation with next generation sequencing (ChIP-Seq) revealed that LSD1 may regulate a subset of genes involved in multiple oncogenic programs to maintain LSC. In some embodiments, the methods, compositions and kits disclosed herein are for treating cancers having stem cell properties, such as AMLs.

LSD1 has been found to possess oncogenic properties in various cancers, such as prostate cancer, bladder cancer, neuroblastomas, lung cancer, sarcomas, and hepato-carcinomas. Overexpression of LSD1 is observed in bladder cancer, lung cancer (e.g., NSCLC), breast cancer, ovary cancer, glioma, colorectal cancer, sarcoma, neuroblastoma, prostate cancer, esophageal squamous cell carcinoma, papillary thyroid carcinoma, and other cancers. Overexpression of LSD1 is found to be associated with clinically aggressive cancers such as recurrent prostate cancer, NSCLC, glioma, breast, colon cancer, ovary cancer, esophageal squamous cell carcinoma, and neuroblastoma. Knockdown of LSD1 expression or treatment with small molecular LSD1 inhibitors results in decreased cancer cell proliferation and/or induction of apoptosis. LSD1 pharmacological inhibitors have been shown, for example, to treat leukemias and also solid tumors. Many natural and synthetic LSD1 inhibitors have been identified, some of which currently undergo clinical assessment.

Natural LSD1 inhibitors include, but are not limited to, cyclic peptides, protoberberine alkaloids, flavones, xanthones, stilbenes, diarylheptanoids, melatonin, and oleacein. In some embodiments, the natural LSD1 inhibitor is capsaicin, Biochanin A, Salvianolic acid B, rosmarinic acid, dihydrotanshinone I, cryptotanshinone, tanshinone I, and an isoquinoline alkaloid (e.g., epiberberine, columbamine, jatrorrhizine, berberine, and palmatine).

The LSD1 inhibitor can covalently or non-covalently bind to LSD1 or FAD and thus modifying the activity by steric hindrance or modification. The LSD1 inhibitors include small molecules inhibiting LSD1 demethylase activity, nucleic acid molecules interfering specifically with LSD1 expression (e.g., RNAi, antisense or ribozyme molecules), substrate-like peptide inhibitors (i.e. a bait-substrate), and aptamers or antibodies directed against LSD1.

The LSD1 inhibitor can be a nucleic acid molecule interfering specifically with LSD1 expression, including for example, RNAi, antisense and ribozyme molecules. A nucleic acid molecule interfering with LSD1 expression is a nucleic acid molecule which is able to reduce or to suppress the expression of the KDM1A gene, in a specific way. As used herein, the term “RNAi” refers to any RNA which is capable of down-regulating the expression of the targeted protein. It encompasses small interfering RNA (siRNA), double-stranded RNA (dsRNA), single-stranded RNA (ssRNA), micro-RNA (miRNA), and short hairpin RNA (shRNA) molecules. Examples of such interfering nucleic acid molecules include, but are not limited to, shRNAs targeting LSD1, miRNA-137 repressing LSD1 expression by targeting its 3′ UTR, and a siRNA targeting LSD1.

The LSD1 inhibitor can be a bait-substrate which is a substrate-like peptide inhibitor, i.e. a peptide which is able to bind to LSD1 and thus prevent interaction of LSD1 with its substrate, in particular with histone H3. Examples of such bait-substrates include, but are not limited to, a bait-substrate derived from the N-terminal 21 amino-acid residues of histone H3 peptide in which lysine 4 is replaced by methionine. This bait binds to LSD1 with high binding affinity and acts as an inhibitor. In some embodiments, the LSD1 inhibitor is a histone H3 peptide based LSD1-selective inhibitor, including but not limited to a peptide having a phenylcyclopropylamine (PCPA) moiety at Lys-4 of the 21 amino acid residues of histone H3 (T. Kakizawa et al. Bioorg. Med. Chem. Lett. 25 (2015) 1925-1928).

The LSD1 inhibitor can be an antibody directed against LSD1. The LSD1 inhibitor can be an aptamer directed against LSD1. The aptamer can be a peptide aptamer or a nucleic acid aptamer. Peptides aptamers consist of a short variable peptide loop attached at both ends to a protein scaffold such as the bacterial protein thioredoxin-A. Typically, the variable loop length is composed of ten to twenty amino acids.

The LSD1 inhibitor can be a small molecule inhibiting LSD1 demethylase activity. The small molecule can be an organic or inorganic compound, usually less than 1000 Daltons, with the ability to inhibit or reduce the activity of LSD1. The small molecule can be derived from any known organism (e.g., animals, plants, bacteria, fungi and viruses) or from a library of synthetic molecules. Exemplary small molecule LSD1 inhibitors include, but are not limited to, cyclopropylamide-based LSD1 inhibitors, peptide-based inhibitors such as described by Culhane et al. (J Am Chem Soc. 2006 Apr. 12; 128(14):4536-7, and J. Am. Chem. Soc., 2010, 132 (9), pp 3164-3176), phenelzine and its analogues (Culhane et al., J. Am. Chem. Soc., 2010, 132 (9), pp 3164-3176, Prusevich et al. ACS Chem. Biol., 2014, 9 (6), pp 1284-1293), polyamine analogues such as the polyamine analogue 2d (1,15-bis {N(5)-[3,3-(diphenyl)propyl]-N(1)-biguanido}-4,12-diazapentadecane) and polyamine analogues described by Huang et al. (Proc Natl Acad Sci USA. 2007 May 8; 104(19):8023-8, and Clin Cancer Res. 2009 Dec. 1; 15(23):7217-28), isosteric ureas and thioureas such as described by Sharma et al. (J. Med. Chem., 2010, 53 (14), pp 5197-5212), chemical inhibitors exhibiting a guanidinium group which mimics arginines and form strong hydrogen bonds with the negatively charged residues of LSD1 (Wang et al., Cancer Res. 2011 71(23): 7238-7249; in particular CBB1002, CBB1003, CBB1004, CBB1005, CBB1006, CBB1007 and CBB1008), namoline (Willmann et al., Int J Cancer. 2012 Dec. 1; 131(11):2704-9; Schmitt et al. J. Med. Chem. 2013, 56(18), 7334-7342), SP2509 (CAS number: 1423715-09-6, Sankar et al. Clin. Cancer Res. 20(17), 4584-4597 (2014) and Fiskus et al. Leukemia 28(11), 2155-2164 (2014)) and pargyline (CAS number: 555-57-7, Wand et al. Biochemical and Biophysical Research Communications (2015)).

Non-limiting examples of LSD1 inhibitor include cyclopropylamide-based LSD1 inhibitors, peptide-based inhibitors, phenelzine and its analogues (e.g., bizine (CAS number: 1591932-50-1)), isosteric ureas, or thioureas. The cyclopropylamine-based LSD1 inhibitor can be, for example, 2-phenylcyclopropan-1-amine and derivatives thereof, and substituted trans-2-arylcyclopropylamines such as described by Gooden et al (Bioorg Med Chem Lett 2008; 18:3047-51). In some embodiments, the LSD1 inhibitor is a 2-phenylcyclopropan-1-amine or a derivative thereof; or a trans-2-phenylcyclopropan-1-amine (tranylcypromine) or a derivatives thereof. In some embodiments, the LSD1 inhibitor is a chemical inhibitor exhibiting a guanidinium group, namoline, SP2509, or pargyline.

In some embodiments, the LSD1 inhibitor is a small molecule selected from tranylcypromine, RN-1 (hydrochloride) (CAS number: 1781835-13-9), GSK LSD1 dihydrochloride (CAS number: 1431368-48-7), OG-L002 (CAS number: 1357302-64-7), trans-N-((2,3-dihydrobenzo[b](1,4]dioxin-6-yl)methyl)-2-phenylcyclopropan-1-amine (Lynch et al. Expert Opin. Ther. Targets (2012) 16(12):1239-1249), trans-N-((2-methoxypyridin-3-yl)methyl)-2-phenylcyclopropan-1-amine (Lynch et al. Expert Opin. Ther. Targets (2012) 16(12):1239-1249), ORY-1001 (CAS number: 1431326-61-2), OG86 (also known as compound B), GSK2879552 (CAS number: 1401966-69-5), ORY-2001 (Oryzon Genomics) and compounds 1, 2, 3 and 4 described in Feng et al. (Journal of Hematology & Oncology (2016) 9:24), or a pharmaceutically acceptable salt, hydrate, solvate or prodrug thereof.

The synthetic LSD1 inhibitors can include tetrahydroquinolines, thieno[3,2-b]pyrroles, tranylcypromines, [1-3]triazolo[4,5-d]pyrimidines, 5-cyano-6-phenylpyrimidines, 7-oxabicyclo[2.2.1]hept-5-ene-2-sulfonates, or 4-hydroxy-3-methylbenzofuran-2-carbohydrazones.

Non-limiting examples of LSD1 inhibitors include tranylcypromine and derivatives thereof, bizine, RN-1 (hydrochloride), GSK LSD1 dihydrochloride, OG-L002, trans-N-((2,3-dihydrobenzo[b](1,4]dioxin-6-yl)methyl)-2-phenylcyclopropan-1-amine, trans-N-((2-methoxypyridin-3-yl)methyl)-2-phenylcyclopropan-1-amine, ORY-1001, OG86, GSK2879552, IMG-7289, INCB059872, CC-90011, ORY-2001, MC2580, DDP38003, (R)-4-[5-(Pyrrolidin-3-ylmethoxy)-2-p-tolyl-pyridin-3-yl]-benzonitrile, 1-(4-methyl-1-piperazinyl)-2-[[(1R*,2S*)-2-[4-phenylmethoxy)phenyl]cyclopropyl]amino]ethanone, N-[4-[trans-2-aminocyclopropyl]phenyl]-4-(4-methylpiperazin-1-yl)benzamide, namoline, pargyline, SP2509, S1201, S2101, C76, GSK690, Cpd 2d, RN7, RO7051790, SYHA1807, TAS1440, SP-2577 (seclidemstat), 2-PFPA, NCL-1, or a pharmaceutically acceptable salt, hydrate, solvate or prodrug thereof.

The LSD1 inhibitors can be an irreversible or a reversible inhibitor to LSD1. In some embodiments, the LSD inhibitor is an irreversible inhibitor, including but not limited to, tranylcypromine (TCP), ORY-1001, ORY-2001, GSK2879552, IMG-7289, INCB059872, and T3775440, and reversible inhibitors, such as SP2509 and CC-90011. Of these, The MAO inhibitor tranylcypromine (TCP) was initially approved by the US Food and Drug Administration (FDA) to treat mood and anxiety disorders and subsequently was found to be able to moderately inhibit its homolog LSD1 by forming a covalent adduct with the flavin ring. Some of the identified LSD1 inhibitors such as TCP, ORY-1001, GSK-2879552, IMG-7289, INCB059872, CC-90011, and ORY-2001 were tested in clinical assessment for cancer therapy, particularly for small lung cancer cells (SCLC) and AML.

The LSD1 inhibitors can also include synthetic molecules as disclosed in, for example, U.S. Pat. Nos. 11,168,082, 11,155,532, 11,013,718, and 10,723,742 and U. S. Patent Application Publication Nos. 2021/0179634, 2021/0179603, 2021/0100800, 2021/0009511, 2020/0392143, 2020/0289437, and 2020/0054578, the contents of which are incorporated herein by reference in their entirety.

In some embodiments, the LSD1 inhibitor is ORY-1001 (Iadademstat, RG-6016, CAS Reg. No. 1431304-21-0, or (trans)-N1-((1R,2S)-2-phenylcyclopropyl)cyclohexane-1,4-diamine), or a pharmaceutically acceptable salt thereof. For example, the LSD1 inhibitor can be ORY-1001 dihydrochloride salt (CAS Reg. No. 1431303-72-8). In a Phase I first-in-human clinical trial, ORY-1001 exhibited a good safety profile and signs of clinical and biologic activity as a single agent in patients with relapsed/refractory acute myeloid leukemia (R/R AML) (EudraCT No.: 2013-002447-29; Salamero et al., J. Clin. Oncol., 2020, 38:4260-4273). In some embodiments, ORY-1001 is administered orally.

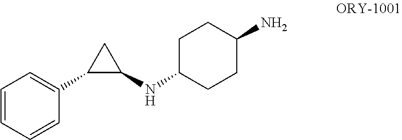 [SMILES: N[C@H]1CC[C@H](N[C@@H]2C[C@H]2c2ccccc2)CC1]

An LSD1 inhibitor can be administered by any suitable routes, including but not limited to oral, topical (including buccal and sublingual), rectal, vaginal, transdermal, parenteral, subcutaneous, intraperitoneal, intrapulmonary, intradermal, intrathecal, epidural, and intranasal administration. Parenteral administration (e.g., injection) can include intramuscular, intravenous, intraarterial, intraperitoneal, or subcutaneous administration.

### PLK1 Inhibitors

Polo-like kinases (PLK) are a family of five highly conserved serine/threonine protein kinases. PLK1 is a master regulator of mitosis and is involved in several steps of the cell cycle, including mitosis entry, centrosome maturation, bipolar spindle formation, chromosome separation, and cytokinesis. PLK1 has been shown to be overexpressed in solid tumors and hematologic malignancies, including AML. PLK1 inhibition induces G2-M-phase arrest with subsequent apoptosis in cancer cells, and has emerged as a promising targeted therapy. Several PLK inhibitors have been studied in clinical trials. In a randomized phase II study of patients with AML who were treatment naïve yet unsuitable for induction therapy, the pan-PLK inhibitor, volasertib (BI6727), administered intravenously in combination with LDAC showed a significant increase in OS when compared with LDAC alone. A subsequent randomized phase III study identified no benefit of the combination and described an increased risk of severe infections. PLK1 facilitates HR during Double Strand DNA Break (DSB) Repair. PLK1 phosphorylates Rad51 and BRCA1, facilitating their recruitment to DSB sites and thereby HR-mediated DNA repair.

Onvansertib (also known as PCM-075, NMS-1286937, NMS-937, “compound of formula (I)” in U.S. Pat. No. 8,927,530; IUPAC name 1-(2-hydroxyethyl)-8-{[5-(4-methylpiperazin-1-yl)-2-(trifluoromethoxy) phenyl] amino}-4,5-dihydro-1H-pyrazolo[4,3-h]quinazoline-3-carboxamide) is a selective ATP-competitive PLK1 inhibitor. Biochemical assays demonstrated high specificity of onvansertib for PLK1 among a panel of 296 kinases, including other PLK members. Onvansertib has potent in vitro and in vivo antitumor activity in models of both solid and hematologic malignancies. Onvansertib is the first PLK1 specific ATP competitive inhibitor administered by oral route to enter clinical trials with proven antitumor activity in different preclinical models. Onvansertib inhibited cell proliferation at nanomolar concentrations in AML cell lines and tumor growth in xenograft models of AML. In addition, onvansertib significantly increased cytarabine antitumor activity in disseminated models of AML.

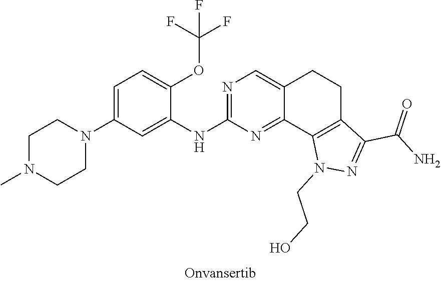 [SMILES: CN1CCN(c2ccc(OC(F)(F)F)c(Nc3ncc4c(n3)-c3c(c(C(N)=O)nn3CCO)CC4)c2)CC1]

Onvansertib shows high potency in proliferation assays having low nanomolar activity on a large number of cell lines, both from solid as well as hematologic tumors. Onvansertib potently causes a mitotic cell-cycle arrest followed by apoptosis in cancer cell lines and inhibits xenograft tumor growth with a clear PLK1-related mechanism of action at well tolerated doses in mice after oral administration. In addition, onvansertib shows activity in combination therapy with approved cytotoxic drugs, such as irinotecan, in which there is enhanced tumor regression in HT29 human colon adenocarcinoma xenografts compared to each agent alone, and shows prolonged survival of animals in a disseminated model of AML in combination therapy with cytarabine. Onvansertib has favorable pharmacologic parameters and good oral bioavailability in rodent and nonrodent species, as well as proven antitumor activity in different nonclinical models using a variety of dosing regimens, which may potentially provide a high degree of flexibility in dosing schedules, warranting investigation in clinical settings. Onvansertib has several advantages over volasertib (BI6727, another PLK1 inhibitor), including a higher degree of potency and specificity for the PLK1 isozyme, and oral bioavailability.

A phase I, first-in-human, dose-escalation study of onvansertib in patients with advanced/metastatic solid tumors identified neutropenia and thrombocytopenia as the primary dose-limiting toxicities. These hematologic toxicities were anticipated on the basis of the mechanism of action of the drug and were reversible, with recovery occurring within 3 weeks. The half-life of onvansertib was established between 20 and 30 hours. The oral bioavailability of onvansertib plus its short half-life provide the opportunity for convenient, controlled, and flexible dosing schedules with the potential to minimize toxicities and improve the therapeutic window. Pharmacodynamics and biomarker studies, including baseline genomic profiling, serial monitoring of mutant allele fractions in plasma, and the extent of PLK1 inhibition in circulating blasts, have been performed to identify biomarkers associated with clinical response and are described in WO2021/146322, the content of which is incorporated herein by reference in its entirety.

As disclosed herein, a combinational therapy using a LSD1 inhibitor and a PLK1 inhibitor (including onvansertib) is expected to result in significantly enhanced efficacy against cancer (e.g., prostate cancer, head and neck cancer, non-small cell lung cancer, intrahepatic cholangiocarcinoma, gastric cancer, urothelial cancer, small cell lung cancer, breast cancer, endometrial cancer, cervical cancer, rhabdomyosarcoma, cholangiocarcinoma, ovarian cancer, or a combination thereof), causing tumor regression and cancer survival. The resulted tumor regression and cancer survival rate/duration by the combination can be surprisingly synergistic (i.e., more than additive, superior to the cumulated anti-tumor efficacy caused by the LSD1 inhibitor and the PLK1 inhibitor separately). The PLK1 inhibitor can be onvansertib. Provided herein include methods, compositions and kits for treating cancer in a subject (for example, a human patient suffering from cancer). The method comprises administrating a LSD1 inhibitor and a PLK1 inhibitor to the patient in a manner sufficient to inhibit or reduce progression of the cancer. For example, the LSD1 inhibitor and the PLK1 inhibitor can be administrated to a subject with cancer simultaneously, separately, or sequentially. It is expected that combination treatment using onvansertib and LSD1 inhibitor is significantly more effective than the combination treatment using another PLK inhibitor BI2536 and LSD1 for various cancer treatment, including the treatment for prostate cancer and lung cancer (e.g., neuroendocrine prostate cancer). It is also expected that the combination treatment using onvansertib and a LSD1 inhibitor has a better safety and toxicity profile than the combination treatment using BI2536 and the LSD1 inhibitor.

In some embodiments, the inhibition or reduction of cancer progression is not merely additive, but is enhanced or synergistic (that is, the inhibition is greater than the combined inhibition of progression caused by the LSD1 inhibitor alone plus the PLK1 inhibitor alone). The enhanced or synergistic efficacy or inhibition of any combination of a LSD1 inhibitor and a PLK1 inhibitor of the present disclosure can be different in different embodiments. In some embodiments, the enhanced or synergistic efficacy or inhibition of any combination of a LSD1 inhibitor and a PLK1 inhibitor of the present disclosure is, is about, is at least, is at least about, is at most, or is at most about, 5%, 10%, 15%, 20%, 25%, 30%, 35%, 40%, 45%, 50%, 55%, 60%, 65%, 70%7, 75%, 80%, 85%, 90%, 95%, 100%, 110%, 120%, 130%, 140%, 150%, 160%, 170%, 180%, 190%, 200%, 210%, 220%, 230%, 240%, 250%, 260%, 270%, 280%, 290%, 300%, or a number or a range between any two of these values, higher than the combined inhibition of progression caused by the LSD1 inhibitor alone plus the PLK1 inhibitor alone.

The molar ratio of the PLK1 inhibitor (e.g., onvansertib) to the LSD1 inhibitor (e.g., ORY-1001) can be, for example, about 1:200, 1:100, 1:90, 1:80, 1:70, 1:60, 1:50, 1:40, 1:30, 1:20, 1:10, 1:1, 10:1, 20:1, 30:1, 40:1, 50:1, 100:1, 1000:1, 2000:1, or 5000:1, or a number or a range between any two of these values. In some embodiments, the enhanced or synergistic efficacy or inhibition of cancer progression caused by a combination of the LSD1 inhibitor (e.g., ORY-1001) and the PLK1 inhibitor (e.g., onvansertib) is, is about, is at least, is at least about, is at most, or is at most about 20%, 30%, 40%, 50%, 60%, 70%, 80%, 90%, 100%, 110%, 120%, 130%, 140%, 150%, 160%, 170%, 180%, 190%, 200%, 250%, 300%, or a number or a range between any two of these values, higher than the combined inhibition of progression caused by the LSD1 inhibitor (e.g., ORY-1001) alone plus the PLK1 inhibitor (e.g., onvansertib) alone. For example, a combination of the LSD1 inhibitor and the PLK1 inhibitor can cause a 50%, 60%, 70%, 80%, 90%, or more, inhibition of cancer progression (cancer cell viability of 50%, 40%, 30%, 20%, 10%, or less), whereas under the same conditions the combined inhibition of the LSD1 inhibitor alone plus the PLK1 inhibitor alone can be 10%, 20%, 25%, 30%, or less) inhibition of cancer progression (cancer cell viability of 90%, 80%, 75%, 70%, or more). Thus, the enhanced or synergistic efficacy or inhibition of cancer progression caused by the combination of the LSD1 inhibitor and the PLK1 inhibitor for example, 50%, 60%, 70%, 80%, 90%, 100%, or more higher than the combined inhibition of progression caused by the LSD1 inhibitor alone plus the PLK1 inhibitor alone. In some embodiments, the LSD1 inhibitor is ORY-1001 and the PLK1 inhibitor is onvansertib.

The method disclosed herein is expected to be effective with various cancer, for example, head and neck cancer, non-small cell lung cancer, intrahepatic cholangiocarcinoma, gastric cancer, urothelial cancer, small cell lung cancer, breast cancer, endometrial cancer, cervical cancer, rhabdomyosarcoma, cholangiocarcinoma, liver cancer, ovarian cancer, prostate cancer, colorectal cancer, pancreatic cancer, prostate cancer, or a combination thereof.

As described herein, the patient can achieve complete response or partial response after treatment with the LSD1 inhibitor and the PLK1 inhibitor. In some embodiments, the patient achieves a complete response. In some embodiments, the patient achieves a partial response. In some embodiments, the patient did not respond to treatment with LSD1 inhibitor(s) (without a PLK1 inhibitor). In some embodiments, the patient did not respond to treatment with the LSD1 inhibitor alone.

The LSD1 inhibitor and the PLK1 inhibitor can be administered to the patient in any manner deemed effective to treat the cancer. The LSD1 inhibitor can be administered together with, or separately from, the PLK1 inhibitor. When administered separately, the LSD1 inhibitor can be administered before or after the PLK1 inhibitor, or in different administration cycles. The LSD1 inhibitor and the PLK1 inhibitor can each be administered in any schedule, e.g., once or multiple times per day or week; once, twice, three times, four times, five times, six times or seven times (daily) per week; for one or multiple weeks; etc. In some embodiments, the LSD1 inhibitor and the PLK1 inhibitor are each administered to the patient in a cycle of at least twice within a week. In other embodiments, the LSD1 inhibitor and the PLK1 inhibitor are each administered to the patient in a cycle of at least five times within a week. In further embodiments, the patient undergoes at least two cycles of administration.

The LSD1 inhibitor can be administered to the patient at any appropriate dosage, e.g., a dosage of about, at least or at most 0.1 μg/kg, 1 μg/kg, 2 μg/kg, 3 μg/kg, 4 μg/kg, 5 μg/kg, 6 μg/kg, 7 μg/kg, 8 μg/kg, 9 μg/kg, 10 μg/kg, 20 μg/kg, 30 μg/kg, 40 μg/kg, 50 μg/kg, 60 μg/kg, 70 μg/kg, 80 μg/kg, 90 μg/kg, 100 μg/kg, 200 μg/kg, 300 μg/kg, 400 μg/kg, 500 μg/kg, 600 μg/kg, 700 μg/kg, 800 μg/kg, 900 μg/kg, 1000 μg/kg (1 mg/kg), 2 mg/kg, 5 mg/kg, 10 mg/kg, 20 mg/kg, or a number between any two of these values. The dosage unit based on the body weight (mg/kg) can be converted to another unit (e.g., mg/m²) using a conversion chart such as the body surface area (BSA) conversion chart as will be understood by a person skilled in the art. In some embodiments, the LSD1 inhibitor is ORY-1001, which is administered at a dosage of about, at least or at most 0.1 μg/kg, 1 μg/kg, 2 μg/kg, 3 μg/kg, 4 μg/kg, 5 μg/kg, 6 μg/kg, 7 μg/kg, 8 μg/kg, 9 μg/kg, 10 μg/kg, 20 μg/kg, 30 μg/kg, 40 μg/kg, 50 μg/kg, 60 μg/kg, 70 μg/kg, 80 μg/kg, 90 μg/kg, 100 μg/kg, 200 μg/kg, 300 μg/kg, 400 μg/kg, 500 μg/kg, or a number between any two of these values.

The LSD1 inhibitor can be administrated to the patient once daily or twice daily. In some embodiments, the LSD1 inhibitor is administered in a cycle of 3-10 days of daily administration. In some embodiments, the LSD1 inhibitor is administered in a cycle of 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, or 30 days. In some embodiments, the LSD1 inhibitor is administered in 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, or 30 days, of a cycle. In some embodiments, the LSD1 inhibitor is administered in day 1, day 2, day 3, day 4, day 5, day 6, day 7, day 8, day 9, day 10, day 11, day 12, day 13, day 14, day 15, day 16, day 17, day 18, day 19, day 20, day 21, day 22, day 23, day 24, day 25, day 26, day 27, day 28, day 29, and/or day 30. In some embodiments, the LSD1 inhibitor is not administered in day 1, day 2, day 3, day 4, day 5, day 6, day 7, day 8, day 9, day 10, day 11, day 12, day 13, day 14, day 15, day 16, day 17, day 18, day 19, day 20, day 21, day 22, day 23, day 24, day 25, day 26, day 27, day 28, day 29, and/or day 30. For example, ORY-1001 can be administered in a cycle of 5, 6, 7, 8, 9, or 10 days. ORY-1001 can be administrated daily on each day or on selected days of the administration cycle. In some embodiments, ORY-1001 is administered in a cycle of 7 days with a daily administration for 5 days (e.g., Days 1-5) and no administration for two days (e.g. Days 6-7).

Similarly, any PLK1 inhibitor, now known or later discovered, can be used in these methods, including PLK1 inhibitors that are selective for PLK1, and PLK1 inhibitors that also inhibit the activity of other proteins. In some embodiments, the PLK1 inhibitor is a dihydropteridinone, a pyridopyrimidine, a aminopyrimidine, a substituted thiazolidinone, a pteridine derivative, a dihydroimidazo[1,5-f]pteridine, a metasubstituted thiazolidinone, a benzyl styryl sulfone analogue, a stilbene derivative, or a combination thereof. In some of these embodiments, the PLK1 inhibitor is onvansertib, BI2536, Volasertib (BI 6727), GSK461364, AZD1775, CYC140, HMN-176, HMN-214, rigosertib (ON-01910), MLN0905, TKM-080301, TAK-960 or Ro3280.

In some embodiments, the PLK1 inhibitor is onvansertib. In these embodiments, the onvansertib is administered to the patient at any appropriate dosage, e.g., a dosage of less than 12 mg/m², less than or equal to 24 mg/m², or greater than 24 mg/m². In particular embodiments, the onvansertib is administered to the patient daily. In additional embodiments, the onvansertib is administered in a cycle of 3-10 days of daily onvansertib administration with 2-16 days with no onvansertib administration.

The combination treatment with onvansertib and LSD1 inhibitor can be administered at the same dose as single treatment with onvansertib or LSD1 inhibitor.

In some embodiments, a PLK1 inhibitor alone or in combination with a LSD1 inhibitor is administrated to a patient who has taken a drug holiday after undergoing one or more cycles of administration. A drug holiday as used herein refers to a period of time when a patient stops taking a PLK1 inhibitor and/or a LSD1 inhibitor. A drug holiday can be a few days to several months. In some embodiments, the drug holiday is, or is about, 1 week, 2 weeks, 3 weeks, 4 weeks, 5 weeks, 6 weeks, 7 weeks, 8 weeks, 9 weeks, 10 weeks, or any value or a range between any two of these values.

As can be appreciated by one of skill in the art, the amount of co-administration of the LSD1 inhibitor and the PLK1 inhibitor, and the timing of co-administration, can depend on the type (species, gender, age, weight, etc.) and condition of the subject being treated and the severity of the disease or condition being treated. The LSD1 inhibitor and the PLK1 inhibitor can formulated into a single pharmaceutical composition, or two separate pharmaceutical compositions. The active ingredients may also be entrapped in microcapsules prepared, for example, by coacervation techniques or by interracial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions.

Methods, compositions, kits and systems disclosed herein can be applied to different types of subjects. For example, the subject can be a subject receiving a cancer treatment, a subject at cancer remission, a subject has received one or more cancer treatment, or a subject suspected of having cancer. The subject can have a stage I cancer, a stage II cancer, a stage III cancer, and/or a stage IV cancer. The cancer can be head and neck cancer, non-small cell lung cancer, intrahepatic cholangiocarcinoma, gastric cancer, urothelial cancer, small cell lung cancer, endometrial cancer, cervical cancer, rhabdomyosarcoma, cholangiocarcinoma, liver cancer, ovarian cancer, breast cancer, prostate cancer, colorectal cancer, pancreatic cancer, or a combination thereof. The cancer can be an LSD1-altered cancer, such as LSD-amplified cancer. The methods can further comprise administering an additional therapeutic intervention to the subject. The additional therapeutic intervention can comprise a different therapeutic intervention than administering the PLK1 inhibitor and the LSD1 inhibitor, an antibody, an adoptive T cell therapy, a chimeric antigen receptor (CAR) T cell therapy, an antibody-drug conjugate, a cytokine therapy, a cancer vaccine, a checkpoint inhibitor, a radiation therapy, surgery, a chemotherapeutic agent, or any combination thereof. The therapeutic intervention can be administered at any time of the treatment, for example at a time when the subject has an early-stage cancer, and wherein the therapeutic intervention is more effective that if the therapeutic intervention were to be administered to the subject at a later time. Without being bound to any particular theory, it is believe that the PLK1 inhibitor (e.g., onvansertib) can sensitize cells (e.g., cancer cells) to LSD1 inhibitor treatment to achieve effective cancer treatment.

### Dosing and Pharmacokinetics

The treatment of the present disclosure can comprise administration of a PLK1 inhibitor (e.g., onvansertib) for a desired duration in one or more cycles of treatment, and administration of an LSD1 inhibitor.

Daily administration of an LSD1 inhibitor (e.g., oral administration) can be at, or be about, 0.01 mg, 0.05 mg, 0.1 mg, 0.15 mg, 0.2 mg, 0.25 mg, 0.3 mg, 0.35 mg, 0.4 mg, 0.45 mg, 0.5 mg, 0.55 mg, 0.6 mg, 0.65 mg, 0.7 mg, 0.75 mg, 0.8 mg, 0.85 mg, 0.9 mg, 0.95 mg, 1 mg, 5 mg, 10 mg, 20 mg, 50 mg, 100 mg, 200 mg, 300 mg, 400 mg, 500 mg, 600 mg, 700 mg, 800 mg, 900 mg, 1000 mg, 1100 mg, 1200 mg, or a number or a range between any two of these values. In some embodiments, the daily dose of the LSD1 inhibitor can be adjusted (e.g., increased or decreased with the range) during the treatment of the subject. The daily administration of the LSD1 inhibitor can be at different amounts on different days or during different weeks. For example, the treatment can comprise daily administration of the LSD1 inhibitor at 0.1 mg to 20 mg during week 1, 0.25 mg to 50 mg during week 2, 0.5 mg to 100 mg during week 3, 1 mg to 200 mg during week 4, and 2 mg to 400 mg during week 5 and beyond. For example, the treatment can comprise daily administration of the LSD1 inhibitor at 0.1 mg to 100 mg on day 1, 0.2 mg to 200 mg on day 2, 0.4 mg to 400 mg on day 3, and 0.4 mg to 400 mg or 0.6 mg to 600 mg on day 4 and beyond. For example, the LSD1 inhibitor is ORY-1001 and is administered at a daily dose of about 0.01 mg, 0.05 mg, 0.1 mg, 0.15 mg, 0.2 mg, 0.25 mg, 0.3 mg, 0.35 mg, 0.4 mg, 0.45 mg, 0.5 mg, 0.55 mg, 0.6 mg, 0.65 mg, 0.7 mg, 0.75 mg, 0.8 mg, 0.85 mg, 0.9 mg, 0.95 mg, 1 mg, 2 mg, 3 mg, 4 mg, 5 mg, 6 mg, 7 mg, 8 mg, 9 mg, 10 mg, or a number or a range between any two of these values. In some embodiments, The daily dose of the LSD1 inhibitor (e.g., ORY-1001) can be, or be about, 0.005 mg/m², 0.01 mg/m², 0.05 mg/m², 0.1 mg/m², 0.15 mg/m², 0.2 mg/m², 0.25 mg/m², 0.3 mg/m², 0.35 mg/m², 0.4 mg/m², 0.45 mg/m², 0.5 mg/m², 0.55 mg/m², 0.6 mg/m², 0.65 mg/m², 0.7 mg/m², 0.75 mg/m², 0.8 mg/m², 0.85 mg/m², 0.9 mg/m², 0.95 mg/m², 1 mg/m², 2 mg/m², 3 mg/m², 4 mg/m², 5 mg/m², 6 mg/m², 7 mg/m², 8 mg/m², 9 mg/m², 10 mg/m², or a number or a range between any two of these values. For example, the LSD1 inhibitor is ORY-1001 and is administered orally at a daily dose of about 0.005 mg/m² to 1 mg/m², such as about 0.005 mg/m² to 0.5 mg/m², about 0.05 mg/m² to 0.25 mg/m², or about 0.05 mg/m² to 0.2 mg/m².

A maximum concentration (Cₘₐₓ) of the LSD1 inhibitor in a blood of the subject (during the treatment or after the treatment) when the LSD1 inhibitor is administered alone or in combination with the PLK1 inhibitor can be from about 1 pg/mL (picogram per mL) to about 10 μg/mL (microgram per mL). For example, the Cₘₐₓ of the LSD1 inhibitor in a blood of the subject when the LSD1 inhibitor is administered alone or in combination with the PLK1 inhibitor can be, or be about, 1 pg/mL, 5 pg/mL, 10 pg/mL, 20 pg/mL, 30 pg/mL, 40 pg/mL, 50 pg/mL, 60 pg/mL, 70 pg/mL, 80 pg/mL, 90 pg/mL, 100 pg/mL, 150 pg/mL, 200 pg/mL, 250 pg/mL, 300 pg/mL, 350 pg/mL, 400 pg/mL, 450 pg/mL, 500 pg/mL, 1000 pg/mL, 5000 pg/mL, 10000 pg/mL, 50000 pg/mL, 100000 pg/mL (0.1 μg/mL), 0.2 μg/mL, 0.3 μg/mL, 0.4 μg/mL, 0.5 μg/mL, 0.6 μg/mL, 0.7 μg/mL, 0.8 μg/mL, 0.9 μg/mL, 1 μg/mL, 1.1 μg/mL, 1.2 μg/mL, 1.3 μg/mL, 1.4 μg/mL, 1.5 μg/mL, 1.6 μg/mL, 1.7 μg/mL, 1.8 μg/mL, 1.9 μg/mL, 2 μg/mL, 2.1 μg/mL, 2.2 μg/mL, 2.3 μg/mL, 2.4 μg/mL, 2.5 μg/mL, 2.6 μg/mL, 2.7 μg/mL, 2.8 μg/mL, 2.9 μg/mL, 3 μg/mL, 3.1 μg/mL, 3.2 μg/mL, 3.3 μg/mL, 3.4 μg/mL, 3.5 μg/mL, 3.6 μg/mL, 3.7 μg/mL, 3.8 μg/mL, 3.9 μg/mL, 4 μg/mL, 4.1 μg/mL, 4.2 μg/mL, 4.3 μg/mL, 4.4 μg/mL, 4.5 μg/mL, 4.6 μg/mL, 4.7 μg/mL, 4.8 μg/mL, 4.9 μg/mL, 5 μg/mL, 5.1 μg/mL, 5.2 μg/mL, 5.3 μg/mL, 5.4 μg/mL, 5.5 μg/mL, 5.6 μg/mL, 5.7 μg/mL, 5.8 μg/mL, 5.9 μg/mL, 6 μg/mL, 6.1 μg/mL, 6.2 μg/mL, 6.3 μg/mL, 6.4 μg/mL, 6.5 μg/mL, 6.6 μg/mL, 6.7 μg/mL, 6.8 μg/mL, 6.9 μg/mL, 7 μg/mL, 7.1 μg/mL, 7.2 μg/mL, 7.3 μg/mL, 7.4 μg/mL, 7.5 μg/mL, 7.6 μg/mL, 7.7 μg/mL, 7.8 μg/mL, 7.9 μg/mL, 8 μg/mL, 8.1 μg/mL, 8.2 μg/mL, 8.3 μg/mL, 8.4 μg/mL, 8.5 μg/mL, 8.6 μg/mL, 8.7 μg/mL, 8.8 μg/mL, 8.9 μg/mL, 9 μg/mL, 9.1 μg/mL, 9.2 μg/mL, 9.3 μg/mL, 9.4 μg/mL, 9.5 μg/mL, 9.6 μg/mL, 9.7 μg/mL, 9.8 μg/mL, 9.9 μg/mL, 10 μg/mL, a range between any two of these values, or any value between 1 μg/mL to 10 μg/mL. For example, the LSD1 inhibitor is ORY-1001, and the Cₘₐₓ of ORY-1001 in a blood of the subject when ORY-1001 is administered alone or in combination with the PLK1 inhibitor can be, or be about, 1 pg/mL, 5 pg/mL, 10 pg/mL, 20 pg/mL, 30 pg/mL, 40 pg/mL, 50 pg/mL, 60 pg/mL, 70 pg/mL, 80 pg/mL, 90 pg/mL, 100 pg/mL, 150 pg/mL, 200 pg/mL, or a number or a range between any two of these values.

An area under curve (AUC) of a plot of a concentration of the LSD1 inhibitor in a blood of the subject over time (e.g., AUC₀₋₂₄ for the first 24 hours after administration) when the LSD1 inhibitor is administered alone or in combination with the PLK1 inhibitor can be from about 1 pg·h/mL to about 100 μg·h/mL. For example, the AUC of a plot of a concentration of the LSD1 inhibitor in a blood of the subject over time (e.g., AUC₀₋₂₄ for the first 24 hours after administration) when the LSD1 inhibitor is administered alone or in combination with the PLK1 inhibitor can be, or be about, 1 pg·h/mL, 5 pg·h/mL, 10 pg·h/mL, 20 pg·h/mL, 30 pg·h/mL, 40 pg·h/ml, 50 pg·h/ml, 60 pg·h/ml, 70 pg·h/ml, 80 pg·h/ml, 90 pg·h/ml, 100 pg·h/ml, 200 pg·h/ml, 300 pg·h/ml, 400 pg·h/ml, 500 pg·h/ml, 600 pg·h/ml, 700 pg·h/ml, 800 pg·h/ml, 900 pg·h/ml, 1000 pg·h/ml, 2000 pg·h/ml, 3000 pg·h/ml, 4000 pg·h/ml, 5000 pg·h/ml, 6000 pg·h/ml, 7000 pg·h/ml, 8000 pg·h/ml, 9000 pg·h/ml, 10000 pg·h/ml, 50000 pg·h/ml, 100000 pg·h/ml, 500000 pg·h/ml, 1000000 pg·h/ml (1 μg·h/mL), 2 μg·h/mL, 3 μg·h/mL, 4 μg·h/mL, 5 μg·h/mL, 6 μg·h/mL, 7 μg·h/mL, 8 μg·h/mL, 9 μg·h/mL, 10 μg·h/mL, 11 μg·h/mL, 12 μg·h/mL, 13 μg·h/mL, 14 μg·h/mL, 15 μg·h/mL, 16 μg·h/mL, 17 μg·h/mL, 18 μg·h/mL, 19 μg·h/mL, 20 μg·h/mL, 21 μg·h/mL, 22 μg·h/mL, 23 μg·h/mL, 24 μg·h/mL, 25 μg·h/mL, 26 μg·h/mL, 27 μg·h/mL, 28 μg·h/mL, 29 μg·h/mL, 30 μg·h/mL, 31 μg·h/mL, 32 μg·h/mL, 33 μg·h/mL, 34 μg·h/mL, 35 μg·h/mL, 36 μg·h/mL, 37 μg·h/mL, 38 μg·h/mL, 39 μg·h/mL, 40 μg·h/mL, 41 μg·h/mL, 42 μg·h/mL, 43 μg·h/mL, 44 μg·h/mL, 45 μg·h/mL, 46 μg·h/mL, 47 μg·h/mL, 48 μg·h/mL, 49 μg·h/mL, 50 μg·h/mL, 51 μg·h/mL, 52 μg·h/mL, 53 μg·h/mL, 54 μg·h/mL, 55 μg·h/mL, 56 μg·h/mL, 57 μg·h/mL, 58 μg·h/mL, 59 μg·h/mL, 60 μg·h/mL, 61 μg·h/mL, 62 μg·h/mL, 63 μg·h/mL, 64 μg·h/mL, 65 μg·h/mL, 66 μg·h/mL, 67 μg·h/mL, 68 μg·h/mL, 69 μg·h/mL, 70 μg·h/mL, 71 μg·h/mL, 72 μg·h/mL, 73 μg·h/mL, 74 μg·h/mL, 75 μg·h/mL, 76 μg·h/mL, 77 μg·h/mL, 78 μg·h/mL, 79 μg·h/mL, 80 μg·h/mL, 81 μg·h/mL, 82 μg·h/mL, 83 μg·h/mL, 84 μg·h/mL, 85 μg·h/mL, 86 μg·h/mL, 87 μg·h/mL, 88 μg·h/mL, 89 μg·h/mL, 90 μg·h/mL, 91 μg·h/mL, 92 μg·h/mL, 93 μg·h/mL, 94 μg·h/mL, 95 μg·h/mL, 96 μg·h/mL, 97 μg·h/mL, 98 μg·h/mL, 99 μg·h/mL, 100 μg·h/mL, or a number or a range between any two of these values, or any value between 1 pg·h/mL and 100 μg·h/mL. For example, the LSD1 inhibitor is ORY-1001, and the AUC of a plot of a concentration of ORY-1001 in a blood of the subject over time (e.g., AUC₀₋₂₄ for the first 24 hours after administration) when ORY-1001 is administered alone or in combination with the PLK1 inhibitor can be, or be about, 1 pg·h/mL, 5 pg·h/mL, 10 pg·h/mL, 20 pg·h/mL, 30 pg·h/mL, 40 pg·h/mL, 50 pg·h/mL, 60 pg·h/mL, 70 pg·h/mL, 80 pg·h/mL, 90 pg·h/mL, 100 pg·h/mL, 200 pg·h/mL, 300 pg·h/mL, 400 pg·h/mL, 500 pg·h/mL, 600 pg·h/mL, 700 pg·h/mL, 800 pg·h/mL, 900 pg·h/mL, 1000 pg·h/mL, 2000 pg·h/mL, 5000 pg·h/mL, or a number or a range between any two of these values.

A time (Tₘₐₓ) to reach a maximum concentration of the LSD1 inhibitor in a blood of the subject when the LSD1 inhibitor is administered alone or in combination with the PLK1 inhibitor can be from about 3 hours to 10 hours. For example, the time (Tₘₐₓ) to reach a maximum concentration of the LSD1 inhibitor in a blood of the subject when the LSD1 inhibitor is administered alone or in combination with the PLK1 inhibitor can be, or be about, 2 hours, 3 hours, 4 hours, 5 hours, 6 hours, 7 hours, 8 hours, 9 hours, 10 hours, 12 hours, 15 hours, 18 hours, 21 hours, 24 hours, a range between any two of these values, or any value between 2 hours and 24 hours. For example, the LSD1 inhibitor is ORY-1001, and the time (Tₘₐₓ) to reach a maximum concentration of ORY-1001 in a blood of the subject when ORY-1001 is administered alone or in combination with the PLK1 inhibitor can be, or be about 4 hours, 5 hours, 6 hours, 8 hours, 12 hours, 18 hours, or a number or a range between any two of these values.

An elimination half-life (T_{1/2}) of the LSD1 inhibitor in a blood of the subject when LSD1 inhibitor is administered alone or in combination with the PLK1 inhibitor can be from about 10 hours to about 100 hours. For example, the elimination half-life (T_{1/2}) of the LSD1 inhibitor in a blood of the subject when the LSD1 inhibitor is administered alone or in combination with the PLK1 inhibitor can be, or be about, 10 hours 15 hours, 20 hours, 25 hours, 30 hours, 35 hours, 40 hours, 45 hours, 50 hours, 55 hours, 60 hours, 65 hours, 70 hours, 75 hours, 80 hours, 85 hours, 90 hours, 95 hours, 100 hours, a range between any two of these values, or any value between 15 hours and 100 hours. For example, the LSD1 inhibitor is ORY-1001, and the elimination half-life (T_{1/2}) of ORY-1001 in a blood of the subject when ORY-1001 is administered alone or in combination with the PLK1 inhibitor can be, or be about, 40 hours, 50 hours, 60 hours, 70 hours, 80 hours, 90 hours, 100 hours, or a number or a range between any two of these values.

The treatment of the present disclosure can comprise administration of a PLK1 inhibitor (onvansertib) for a desired duration in a cycle. The administration of the PLKs inhibitor (and/or the one or more chemotherapeutic agents) can be daily or with break(s) between days of administrations. The break can be, for example, 1 day, 2 days, 3 days, 4 days, 5 days, 6 days, 7 days, 8 days, 9 days, 10 days, 11 days, 12 days, 13 days, 14 days, or more. The administration can be once, twice, three times, four times, or more on a day when the PLK1 inhibitor (and/or the one or more chemotherapeutic agents) is administered to the patient. The administration can be, for example, once every two days, every three days, every four days, every five days, every six days, or every seven days. The length of the desired duration can vary, for example, 1, 2, 3, 4, 5, 6, 7, 8, 9, 10, 11, 12, 13, 14, 15, 16, 17, 18, 19, 20, 21, 22, 23, 24, 25, 26, 27, 28, or more days. Each cycle of treatment can have various lengths, for example, at least 14 days, 15 days, 16 days, 17 days, 18 days, 19 days, 20 days, 21 days, 22 days, 23 days, 24 days, 25 days, 26 days, 27 days, 28 days, or more. For example, a single cycle of the treatment can comprise administration of the PLK1 inhibitor (e.g., onvansertib) and/or the one or more chemotherapeutic agents for four days, five days, six days, seven days, eight days, nine days, ten days, eleven days, twelve days, thirteen days, fourteen days, fifteen days, sixteen days, seventeen days, eighteen days, nineteen days, twenty days, twenty-one days, twenty-two days, twenty-three days, twenty-four days, twenty-five days, twenty-six days, twenty-seven days, twenty-eight days, or more in a cycle (e.g., in a cycle of at least 21 days (e.g., 21 to 28 days)). In some embodiments, the treatment can comprise administration of the PLK1 inhibitor (e.g., onvansertib) and/or the one or more chemotherapeutic agents for, or for at least, four days, five days, six days, seven days, eight days, nine days, ten days, eleven days, twelve days, thirteen days, fourteen days, fifteen days, sixteen days, seventeen days, eighteen days, nineteen days, twenty days, or a range between any two of these values, in a cycle (e.g., a cycle of at least 21 days (e.g., 21 to 28 days)). The administration of the PLK1 inhibitor (e.g., onvansertib) and/or the one or more chemotherapeutic agents in a single cycle of the treatment can be continuous or with one or more intervals (e.g., one day or two days of break). In some embodiments, the treatment comprises administration of the PLK1 inhibitor (e.g., onvansertib) for five days in a cycle of 21 to 28 days.

In some embodiments, the PLK1 inhibitor (e.g., onvansertib) is administered to the subject in need thereof on twenty days (e.g., Days 1-10 and 15-24) during a 28-day cycle. The twenty days can be, for example, a continuous daily administration for ten days (e.g., Days 1-10) and another continuous daily administration (e.g., Days 15-24) for ten days, or a continuous daily administration for four sets of five days (e.g., Days 1-5, 8-12, 15-19, and 22-26), In some embodiments, for example when the patient is identified to have low tolerance to the PLK1 inhibitor (e.g., onvansertib), the PLK1 inhibitor is administered to the subject in need thereof on ten days (e.g., Days 1-5 and 15-19) during a 28-day cycle. The ten days can be, for example, a continuous daily administration for ten days (e.g., Days 1-10) or two continuous daily admiration for five days each (e.g., Days 1-5 and Days 15-19). In some embodiments, the PLK1 inhibitor (e.g., onvansertib) is administered to the subject in need thereof daily throughout the whole cycle (e.g., daily for 28 days in a cycle of 28 days). Depending on the needs of inhibition/reversion of cancer progression in the subject, the subject can receive one, two, three, four, five, six, or more cycles of treatment. For combination treatment, the administration cycles, dosing schedules, and/or dosage amounts of the LSD1 inhibitor and the PLK1 inhibitor can be the same or different. For combination treatment, the administration cycle, dosing schedule, and/or dosage amount of the LSD1 inhibitor can be adjusted according to the administration cycle, dosing schedule, and/or dosage amount of the PLK1 inhibitor. For example, the LSD1 inhibitor (e.g., ORY-1001) can be administered in four 7-day cycles (e.g., daily dose on Days 1-5 and no dose on Days 6-7, repeated for 4 weeks), which corresponds to a 28-day cycle for administration of the PLK1 inhibitor (e.g., onvansertib).

The treatment can comprise administration of the PLK1 inhibitor (e.g., onvansertib) at, or at about, 6 mg/m²-90 mg/m², for example, as a daily dose. For example, the treatment can comprise daily administration of the PLK1 inhibitor (e.g., onvansertib) at, or at about, 6 mg/m², 8 mg/m², 10 mg/m², 12 mg/m², 14 mg/m², 16 mg/m², 18 mg/m², 20 mg/m², 23 mg/m², 27 mg/m², 30 mg/m², 35 mg/m², 40 mg/m², 45 mg/m², 50 mg/m², 55 mg/m², 60 mg/m², 65 mg/m², 70 mg/m², 80 mg/m², 85 mg/m², 90 mg/m², a number or a range between any two of these values, or any value between 8 mg/m²-90 mg/m². In some embodiments, the daily dose of the PLK1 inhibitor (e.g., onvansertib) can be adjusted (e.g., increased or decreased with the range) during the treatment, or during a single cycle (e.g., the first cycle, the second cycle, the third cycle, and a subsequent cycle) of the treatment, for the subject. In some embodiments, the PLK inhibitor (e.g., onvansertib) is administered at 12 mg/m² on twenty days (e.g., Days 1-10 and 15-24) during a 28-day cycle. In some embodiments, the PLK inhibitor (e.g., onvansertib) is administered at 15 mg/m² on ten days (e.g., Days 1-5 and 15-19) during a 28-day cycle. In some embodiments, the PLK inhibitor (e.g., onvansertib) is administered at 8 mg/m² or 10 mg/m² everyday (e.g., Days 11-28) during a 28-day cycle. In some embodiments, the daily dose of the PLK1 inhibitor (e.g., onvansertib) can be adjusted (e.g., increased or decreased with the range) during the treatment, or during a single cycle (e.g., the first cycle, the second cycle, the third cycle, and a subsequent cycle) of the treatment, for the subject.

A maximum concentration (Cₘₐₓ) of the PLK1 inhibitor (e.g., onvansertib) in a blood of the subject (during the treatment or after the treatment) when the PLK1 inhibitor is administered alone or in combination with the LSD1 inhibitor can be from about 100 nmol/L to about 1500 nmol/L. For example, the Cₘₐₓ of the PLK1 inhibitor (e.g., onvansertib) in a blood of the subject when the PLK1 inhibitor is administered alone or in combination with the LSD1 inhibitor can be, or be about, 100 nmol/L, 200 nmol/L, 300 nmol/L, 400 nmol/L, 500 nmol/L, 600 nmol/L, 700 nmol/L, 800 nmol/L, 900 nmol/L, 1000 nmol/L, 1100 nmol/L, 1200 nmol/L, 1300 nmol/L, 1400 nmol/L, 1500 nmol/L, a range between any two of these values, or any value between 200 nmol/L to 1500 nmol/L.

An area under curve (AUC) of a plot of a concentration of the PLK1 inhibitor (e.g., onvansertib) in a blood of the subject over time (e.g., AUC₀₋₂₄ for the first 24 hours after administration) when the PLK1 inhibitor is administered alone or in combination with the LSD1 inhibitor can be from about 1000 nmol/L·hour to about 400000 nmol/L·hour. For example, the AUC of a plot of a concentration of the PLK1 inhibitor (e.g., onvansertib) in a blood of the subject over time (e.g., AUC₀₋₂₄ for the first 24 hours after administration) when the PLK1 inhibitor is administered alone or in combination with the LSD1 inhibitor can be, or be about, 1000 nmol/L·hour, 5000 nmol/L·hour, 10000 nmol/L·hour, 15000 nmol/L·hour, 20000 nmol/L·hour, 25000 nmol/L·hour, 30000 nmol/L·hour, 35000 nmol/L·hour, 40000 nmol/L·hour, a range between any two of these values, or any value between 1000 nmol/L·hour and 400000 nmol/L·hour.

A time (Tₘₐₓ) to reach a maximum concentration of the PLK1 inhibitor (e.g., onvansertib) in a blood of the subject when the PLK1 inhibitor is administered alone or in combination with the LSD1 inhibitor can be from about 1 hour to about 5 hours. For example, the time (Tₘₐₓ) to reach a maximum concentration of the PLK1 inhibitor (e.g., onvansertib) in a blood of the subject when the PLK1 inhibitor is administered alone or in combination with the LSD1 inhibitor can be, or be about, 1 hour, 1.5 hours, 2 hours, 2.5 hours, 3 hours, 3.5 hours, 4 hours, 4.5 hours, 5 hours, a range between any two of these values, or any value between 1 hour and 5 hours.

An elimination half-life (T_{1/2}) of the PLK1 inhibitor (e.g., onvansertib) in a blood of the subject when the PLK1 inhibitor is administered alone or in combination with the LSD1 inhibitor can be from about 10 hours to about 60 hours. For example, the elimination half-life (T_{1/2}) of the PLK1 inhibitor (e.g., onvansertib) in a blood of the subject when the PLK1 inhibitor is administered alone or in combination with the LSD1 inhibitor can be, or be about, 10 hours, 15 hours, 20 hours, 25 hours, 30 hours, 35 hours, 40 hours, 45 hours, 50 hours, 55 hours, 60 hours, a range between any two of these values, or any value between 10 hours and 60 hours.

### Additional Cancer Therapeutics or Therapy

Methods, compositions and kits disclosed herein can be used for treating cancer, for example prostate cancer. In some embodiments, a method for treating cancer comprises administrating an LSD1 inhibitor and a PLK1 inhibitor (e.g., onvansertib) to a subject (e.g., a patient) in need thereof. The method can comprise administering a therapeutically effective amount of the LSD1 inhibitor and a therapeutically effective amount of the PLK1 inhibitor. The treatment can comprise administration of at least one additional cancer therapeutics or cancer therapy. The treatment can comprise administration a therapeutically effective amount of at least one additional cancer therapeutics or cancer therapy. The LSD1 inhibitor and the cancer therapeutics or cancer therapy can, for example, co-administered simultaneously or sequentially. The PLK1 inhibitor (e.g., onvansertib) and the cancer therapeutics or cancer therapy can, for example, co-administered simultaneously or sequentially.

### Methods for Predicting/Determining Treatment Efficacy and Status for Cancer

Also disclosed herein include methods, compositions, kits, and systems for predicting/determining clinical outcome for a combination treatment of cancer of the present disclosure, monitoring of the combination treatment, predicting/determining responsiveness of a subject to the combination treatment, determining the status of the cancer in a subject, and improving combination treatment outcome. The methods, compositions, kits and systems can be used to guide the combination treatment, provide combination treatment recommendations, reduce or avoid unnecessary ineffective combination treatment for patients. ctDNA can be analyzed to predict/determine clinical outcome for cancer treatment using a combination of a LSD1 inhibitor and a PLK1 inhibitor of the present disclosure, monitor the combination treatment, predict/determine responsiveness of a subject to the combination treatment, determine cancer status in a subject, improve combination treatment outcome, guide combination treatment, provide combination treatment recommendations, and/or to reduce or avoid ineffective combination treatment. ctDNA can be analyzed to predict/determine clinical outcome for cancer treatment, monitor cancer treatment, predict/determine responsiveness of a subject to a cancer treatment, determine cancer status in a subject, improve cancer treatment outcome, guide cancer treatment, provide treatment recommendations, and/or to reduce or avoid ineffective cancer treatment. Such analysis of ctDNA has been described in PCT Application No. PCT/US2021/013287, the content of which is incorporated herein by reference in its entirety.

A method of determining responsiveness of a subject to a combination treatment comprising a LSD1 inhibitor and a PLK1 inhibitor of the disclosure can comprise, for example, analyzing circulating tumor DNA (ctDNA) of a subject with cancer, the subject is undergoing a treatment and/or has received the combination treatment, thereby determining the responsiveness of the subject to the combination treatment. In some embodiments, determining the responsiveness of the subject comprises determining if the subject is a responder of the treatment, if the subject is or is going to be in CR, or if the subject is or is going to be in partial remission (PR). For example, analyzing ctDNA can comprise detecting variant allele frequency in the ctDNA in a first sample obtained from the subject at a first time point, detecting variant allele frequency in the ctDNA obtained from the subject at one or more additional time points in one or more additional samples, and determining the difference of the variant allele frequency in ctDNA between the first and at least one of the one or more additional samples, a decrease in the variant allele frequency in at least one of the additional samples relative to the first sample indicates the subject as responsive to the cancer treatment.

In some embodiments, the first time point is prior or immediately prior to the combination treatment, and at least one of the one or more additional time points are at the end of or after at least a cycle of the combination treatment. In some embodiments, the cycle of the combination treatment is the first cycle of the combination treatment. In some embodiments, the first time point is prior or immediately prior to a first cycle of the combination treatment, and the one or more additional time points are at the end of or after a second cycle of the combination treatment.

In some embodiments, the first cycle of the combination treatment is immediately prior to the second cycle of the combination treatment. In some embodiments, the method comprises continuing the combination treatment to the subject if the subject is indicated as responsive to the combination treatment. In some embodiments, the method comprises discontinuing the combination treatment to the subject and/or starting a different combination treatment to the subject if the subject is not indicated as responsive to the combination treatment.

Disclosed herein include methods of determining cancer status of a subject, comprising analyzing circulating tumor DNA (ctDNA) of a subject, thereby determining cancer status of the subject. The subject can be a subject undergoing a current combination treatment comprising a LSD1 inhibitor and a PLK1 inhibitor of the present disclosure, a subject that has received a prior combination treatment of the present disclosure, and/or a subject that is in remission for the cancer. The subject in remission for cancer can be in complete remission (CR), or in partial remission (PR).

In some embodiments, analyzing the ctDNA comprises detecting variant allele frequency in the ctDNA. In some embodiments, analyzing the ctDNA comprises detecting variant allele frequency in the ctDNA obtained from the subject at a first time point in a first sample, detecting variant allele frequency in the ctDNA obtained from the subject at one or more additional time points in one or more additional samples, and determining the difference of the variant allele frequency in ctDNA between the first and at least one of the one or more additional samples, an increase in the variant allele frequency at the additional sample(s) relative to the first sample indicates that the subject is at risk of cancer relapse or is in cancer relapse.

In some embodiments, the first time point is prior or immediately prior to the combination treatment, and the one or more additional time points are at the end of or after at least a cycle of the combination treatment, optionally the cycle of the combination treatment is the first cycle of the combination treatment. In some embodiments, the first time point is prior or immediately prior to a first cycle of the combination treatment, and the one or more additional time points are at the end of or after a second cycle of the combination treatment, optionally the first cycle of the combination treatment is immediately prior to the second cycle of the combination treatment.

In some embodiments, the method comprises starting an additional treatment to the subject if the subject is indicated as in cancer relapse. The additional treatment can be the same or different from the current or prior combination treatment.

The variant allele frequency in ctDNA can be determined, for example, by total mutation count in the ctDNA in each of the first sample and one or more additional samples, or by the mean variant allele frequency in each of the first sample and one or more additional samples. In some embodiments, the variant allele frequency is mutant allelic frequency (MAF) for a driver mutation of the cancer (e.g., ovarian cancer, breast cancer, prostate cancer, colorectal cancer, pancreatic cancer, or a combination thereof). In some embodiments, the variant allele frequency is MAF for one or more driver mutations of the cancer (e.g., ovarian cancer, breast cancer, prostate cancer, colorectal cancer, pancreatic cancer, or a combination thereof). In some embodiments, Log₂(C₁/C₀)<a MAF threshold indicates a decrease in ctDNA MAF C₀ is ctDNA MAF in the first sample and C₁ is ctDNA MAF in one of the additional samples. In some embodiments, the MAF threshold is, or is about, 0.01 to −0.10. In some embodiments, the MAF threshold is, or is about, 0.06. In some embodiments, the MAF threshold is, or is about, 0.05.

In some embodiments, the first sample comprises ctDNA from the subject before treatment, and the one of additional samples comprises ctDNA from the subject after treatment. In some embodiments, the driver mutation is a mutation in one of the below 75 genes ABL1, ANKRD26, ASXL1, ATRX, BCOR, BCORL1, BRAF, BTK, CALR, CBL, CBLB, CBLC, CCND2, CDC25C, CDKN2A, CEBPA, CSF3R, CUX1, CXCR4, DCK, DDX41, DHX15, DNMT3A, ETNK1, ETV6, EZH2, FBXW7, FLT3, GATA1, GATA2, GNAS, HRAS, IDH1, IDH2, IKZF1, JAK2, JAK3, KDM6A, KIT, KMT2A, KRAS, LUC7L2, MAP2K1, MPL, MYC, MYD88, NF1, NOTCH1, NPM1, NRAS, PDGFRA, PH1F6, PPM1D, PTEN, PTPN11, RAD21, RBBP6, RPS14, RUNX1, SETBP1, SF3B1, SH2B3, SLC29A1, SMC1A, SMC3, SRSF2, STAG2, STAT3, TET2, TP53, U2AF1, U2AF2, WT1, XPO1, and ZRSR2. In some embodiments, at least one of the one or more the driver mutations is a mutation in in the 75 genes. In some embodiments, one or more the driver mutations are mutations in the 75 genes.

The driver mutation or at least one of the one or more driver mutations can be in a gene selected from the group consisting of TP53, ASXL1, DNMT3A, NRAS, SRSF2, TET2, SF3B1, FLT3, FLT3 ITD, IDH2, NPM1, RUNX1, CDKN2A, KRAS, STAG2, CALR, CBL, CSF3R, DDX41, GATA2, JAK2, PH1F6, and SETBP1. In some embodiments, the driver mutation or at least one of the one or more driver mutations is in a gene selected from the group consisting of DNMT3A, TET2, NPM1, SRSF2, NRAS, CDKN2A, SF3B1, FLT3, ASXL1, SRSF2, IDH2, NRAS, and SF3B1. In some embodiments, the method further comprises determining variant allele frequency in one or more of the ctDNA, PBMCs and BMMCs of the subject.

The ctDNA can be analyzed using, for example, polymerase chain reaction (PCR), next generation sequencing (NGS), and/or droplet digital PCR (ddPCR). The sample disclosed herein can be derived from, for example, whole blood of the subject, plasma of the subject, serum of the subject, or a combination thereof. In some embodiments, the ctDNA is from whole blood of the subject, plasma of the subject, serum of the subject, or a combination thereof.

In some embodiments, the method comprises analyzing ctDNA of the subject before the treatment. In some embodiments, the treatment comprises one or more cycles, and the ctDNA is analyzed before, during and after each cycle of the treatment. Each cycle of treatment can be at least 21 days. In some embodiments, each cycle of treatment is from about 21 days to about 28 days. In some embodiments, the subject is human.

Disclosed herein include methods of improving treatment outcome for the cancer. The method can comprise: detecting variant allele frequency in circulating tumor DNA (ctDNA) obtained from a subject at a first time point in a first sample before the subject undergoes a combination treatment comprising a LSD1 inhibitor and a PLK1 inhibitor of the present disclosure; detecting variant allele frequency in ctDNA obtained from the subject at one or more additional time points in one or more additional samples after the subject undergoes the combination treatment; determining the difference of the variant allele frequency in ctDNA between the first and at least one of the one or more additional samples, a decrease in the variant allele frequency in at least one of the additional samples relative to the first sample indicates the subject as responsive to the combination treatment; and continuing the combination treatment to the subject if the subject is indicated as responsive to the combination treatment, or discontinuing the combination treatment to the subject and/or starting a different cancer treatment to the subject if the subject is not indicated as responsive to the combination treatment.

Also disclosed herein include methods of treating cancer The method can comprise: administering a combination treatment comprising a LSD1 inhibitor and a PLK1 inhibitor of the present disclosure to a subject in need thereof, determining a decrease, relative to a variant allele frequency in a first sample of the subject obtained at a first time point before the subject receives the combination treatment, in a variant allele frequency in a second sample of the subject obtained at a second time point after the subject receives the combination treatment; and continuing with the combination treatment. In some embodiments, the subject is a subject newly diagnosed with cancer, for example a subject that has not received any prior cancer treatment before the combination treatment. In some embodiments, the subject has received prior cancer treatment and was in remission for the cancer, for example a subject in complete remission (CR), or in partial remission (PR) after receiving the prior combination treatment.

The first time point can be, for example, prior or immediately prior to the combination treatment. The at least one of the one or more additional time points can be, for example, at the end of or after at least a cycle of the combination treatment. In some embodiments, the cycle of the combination treatment is the first cycle of the combination treatment. In some embodiments, the first time point is prior or immediately prior to a first cycle of the combination treatment, and the one or more additional time points are at the end of or after a second cycle of the combination treatment. In some embodiments, the first cycle of the combination treatment is immediately prior to the second cycle of the combination treatment.

The variant allele frequency in ctDNA can be determined, for example, by total mutation count in the ctDNA in each of the first sample and one or more additional samples, and/or by the mean variant allele frequency in each of the first sample and one or more additional samples. In some embodiments, the variant allele frequency is mutant allelic frequency (MAF) for a driver mutation of the cancer (e.g., ovarian cancer, breast cancer, prostate cancer, colorectal cancer, pancreatic cancer, or a combination thereof). In some embodiments, the variant allele frequency is mutant allelic frequency (MAF) for one or more driver mutations of the cancer (e.g., ovarian cancer, breast cancer, prostate cancer, colorectal cancer, pancreatic cancer, or a combination thereof). In some embodiments, Log₂(C₁/C₀)<a MAF threshold indicates a decrease in ctDNA MAF C₀ is ctDNA MAF in the first sample and C₁ is ctDNA MAF in one of the additional samples. In some embodiments, the MAF threshold is −0.05.

The driver mutation can be, for example, a mutation in one of the 75 genes set forth in Table 3, at least one of the one or more the driver mutations is a mutation in one of the below 75 genes ABL1, ANKRD26, ASXL1, ATRX, BCOR, BCORL1, BRAF, BTK, CALR, CBL, CBLB, CBLC, CCND2, CDC25C, CDKN2A, CEBPA, CSF3R, CUX1, CXCR4, DCK, DDX41, DHX15, DNMT3A, ETNK1, ETV6, EZH2, FBXW7, FLT3, GATA1, GATA2, GNAS, HRAS, IDH1, IDH2, IKZF1, JAK2, JAK3, KDM6A, KIT, KMT2A, KRAS, LUC7L2, MAP2K1, MPL, MYC, MYD88, NF1, NOTCH1, NPM1, NRAS, PDGFRA, PHF6, PPM1D, PTEN, PTPN11, RAD21, RBBP6, RPS14, RUNX1, SETBP1, SF3B1, SH2B3, SLC29A1, SMC1A, SMC3, SRSF2, STAG2, STAT3, TET2, TP53, U2AF1, U2AF2, WT1, XPO1, and ZRSR2, and/or one or more the driver mutations are mutations in the 75 genes. In some embodiments, the driver mutation or at least one of the one or more driver mutations is in a gene selected from the group consisting of TP53, ASXL1, DNMT3A, NRAS, SRSF2, TET2, SF3B1, FLT3, FLT3 ITD, IDH2, NPM1, RUNX1, CDKN2A, KRAS, STAG2, CALR, CBL, CSF3R, DDX41, GATA2, JAK2, PH1F6, and SETBP1. In some embodiments, the driver mutation or at least one of the one or more driver mutations is in a gene selected from the group consisting of DNMT3A, TET2, NPM1, SRSF2, NRAS, CDKN2A, SF3B1, FLT3, ASXL1, SRSF2, IDH2, NRAS, and SF3B1.

In some embodiments, the method further comprises determining variant allele frequency in one or more of the ctDNA, PBMCs and BMMCs of the subject. The variant allele frequency in ctDNA can be detected, for example, using polymerase chain reaction (PCR) or next generation sequencing (NGS). In some embodiments, the variant allele frequency in ctDNA is detected using droplet digital PCR (ddPCR).

At least one of the first sample, the one or more additional samples, and the second sample can be derived from whole blood of the subject, plasma of the subject, serum of the subject, or a combination thereof. In some embodiments, the ctDNA is from whole blood of the subject, plasma of the subject, serum of the subject, or a combination thereof.

In some embodiments, the subject whose ctDNA is analyzed is undergoing or will be undergoing treatment for the cancer. The method can comprise analyzing ctDNA of the subject before the treatment. The treatment can comprise one or more cycles, and the ctDNA is analyzed before, during and after one or more cycles of the treatment. For example, the ctDNA can be analyzed before, during and after two or more cycle of the treatment, three or more cycle of the treatment, or each cycle of the treatment. Each cycle of treatment can be at least 21 days, for example, 21 days, 22 days, 23 days, 24 days, 25 days, 26 days, 27 days, 28 days, 29 days, 30 days, or more, or a range between any two of these values. In some embodiments, each cycle of treatment is from about 21 days to about 28 days. In some embodiments, each cycle of treatment is from 21 days to 28 days. In some embodiments, the subject is human.

### Compositions and Kits

Disclosed herein include compositions and kits for treating cancer. In some embodiments, a kit comprises: a Polo-like kinase 1 (PLK1) inhibitor; and a manual providing instructions for co-administrating the PLK1 inhibitor with a LSD1 inhibitor to a subject for treating cancer. In some embodiments, the kit comprises the LSD1 inhibitor. The cancer can be, for example, ovarian cancer, breast cancer, prostate cancer, colorectal cancer, pancreatic cancer, or a combination thereof.

In some embodiments, the subject has cancer (e.g., head and neck cancer, non-small cell lung cancer, small-cell lung cancer, intrahepatic cholangiocarcinoma, gastric cancer, urothelial cancer, breast cancer, endometrial cancer, cervical cancer, rhabdomyosarcoma, cholangiocarcinoma, glioblastoma, low-grade glioma, thyroid carcinoma, gallbladder cancer, ovarian cancer, prostate cancer, or a combination thereof). In some embodiments, the instructions comprise instructions for co-administrating the PLK inhibitor and the LSD1 inhibitor simultaneously. In some embodiments, the instructions comprise instructions for co-administrating the PLK inhibitor and the LSD1 inhibitor sequentially. In some embodiments, the instructions comprise instructions for administering of the PLK1 inhibitor orally. In some embodiments, the instructions comprise instructions for administrating the LSD1 inhibitor orally.

In some embodiments, the instructions comprise instructions the subject has received a prior LSD1 inhibitor treatment. In some embodiments, the instructions comprise instructions the subject did not respond to treatment with the LSD1 inhibitor alone. In some embodiments, the instructions comprise instructions the subject is known to be resistant to a LSD1 inhibitor therapy.

In some embodiments, the instructions comprise instructions the subject has received at least one prior treatment for the cancer. In some embodiments, the prior treatment does not comprise the use of a LSD1 inhibitor, a PLK inhibitor, or both. In some embodiments, the instructions comprise instructions the subject was in remission for the cancer. In some embodiments, the subject in remission for cancer was in complete remission (CR), or in partial remission (PR).

In some embodiments, the instructions comprise instructions for administering each of the LSD1 inhibitor and the PLK1 inhibitor to the subject in a cycle of at least twice within a week. In some embodiments, the instructions comprise instructions for administering each of the LSD1 inhibitor r and the PLK1 inhibitor to the subject in a cycle of at least five times within a week In some embodiments, the instructions comprise instructions for administering the LSD1 inhibitor, the PLK1 inhibitor, or both are in a cycle of at least 7 days. In some embodiments, each cycle of treatment is at least about 21 days. In some embodiments, each cycle of treatment is from about 21 days to about 28 days. In some embodiments, the instructions comprise instructions for administering the PLK1 inhibitor on at least four days in the cycle. In some embodiments, the instructions comprise instructions for not administering the PLK1 inhibitor on at least one day in the cycle. In some embodiments, the instructions comprise instructions for administrating the LSD1 inhibitor daily. In some embodiments, the instructions comprise instructions for administrating the LSD1 inhibitor and the PLK1 inhibitor for at least two cycles.

In some embodiments, the LSD1 inhibitor is tranylcypromine and derivatives thereof, bizine, RN-1 (hydrochloride), GSK LSD1 dihydrochloride, OG-L002, trans-N-((2,3-dihydrobenzo[b](1,4]dioxin-6-yl)methyl)-2-phenylcyclopropan-1-amine, trans-N-((2-methoxypyridin-3-yl)methyl)-2-phenylcyclopropan-1-amine, ORY-1001, OG86, GSK2879552, IMG-7289, INCB059872, CC-90011, ORY-2001, MC2580, DDP38003, (R)-4-[5-(Pyrrolidin-3-ylmethoxy)-2-p-tolyl-pyridin-3-yl]-benzonitrile, 1-(4-methyl-1-piperazinyl)-2-[[(1R*,2S*)-2-[4-phenylmethoxy)phenyl]cyclopropyl]amino]ethanone, N-[4-[trans-2-aminocyclopropyl]phenyl]-4-(4-methylpiperazin-1-yl)benzamide, namoline, pargyline, SP2509, S1201, S2101, C76, GSK690, Cpd 2d, RN7, RO7051790, SYHA1807, TAS1440, SP-2577 (seclidemstat), 2-PFPA, NCL-1, HCI-2509, or a pharmaceutically acceptable salt, hydrate, solvate or prodrug thereof. In some embodiments, the LSD1 inhibitor is ORY-1001 or a pharmaceutically acceptable salt thereof.

The PLK1 inhibitor can be selective and/or specific for PLK1. In some embodiments, the PLK1 inhibitor is a dihydropteridinone, a pyridopyrimidine, a aminopyrimidine, a substituted thiazolidinone, a pteridine derivative, a dihydroimidazo[1,5-f]pteridine, a metasubstituted thiazolidinone, a benzyl styryl sulfone analogue, a stilbene derivative, or any combination thereof. In some embodiments, the PLK1 inhibitor is onvansertib, BI2536, Volasertib (BI 6727), GSK461364, AZD1775, CYC140, HMN-176, HMN-214, rigosertib (ON-01910), MLN0905, TKM-080301, TAK-960 or Ro3280. In some embodiments, the PLK1 inhibitor is onvansertib.

In some embodiments, the instructions comprise instructions for administering the PLK1 inhibitor at 8 mg/m²-90 mg/m². In some embodiments, the instructions comprise instructions for administering the LSD1 inhibitor at 0.01 mg-1200 mg (e.g., daily dose of at 0.01 mg-10 mg administered orally).

The methods, compositions and kits disclosed herein can also be used to sensitize cancer cells to one or more LSD1 inhibitors. The method can comprise contacting cancer cells with a composition comprising a PLK1 inhibitor (e.g., onvansertib), or a pharmaceutically acceptable salt, solvate, stereoisomer thereof, thereby sensitizing the cancer cells to the one or more LSD1 inhibitors. Contacting cancer cells with the composition can occur in vitro, ex vivo, in vivo, or in any combination. In some embodiments, contacting cancer cells with the composition is in a subject's body. In some embodiments, cancer cells are contacted with the composition in a cell culture. The subject can be a mammal, for example a human. The sensitization of the cancer cells can increase the responsiveness of the cancer cells to the one or more LSD1 inhibitors (e.g., ORY-1001) by, or by about, 1%, 5%, 10%, 15%, 20%, 25%, 30%, 35%, 40%, 45%, 50%, 55%, 60%, 65%, 70%, 75%, 80%, 85%, 90%, 95%, or a range between any two of these values. The sensitization of the cancer cells can increase the responsiveness of the cancer cells to the one or more LSD1 inhibitors by at least, or by at least about, 1%, 5%, 10%, 15%, 20%, 25%, 30%, 35%, 40%, 45%, 50%, 55%, 60%, 65%, 70%, 75%, 80%, 85%, 90%, 95%, or a range between any two of these values. The increase of the responsiveness of the cancer cells is, in some embodiments, relative to the untreated cancer cells. The sensitization of the cancer cells can increase the responsiveness of the subject having the cancer cells to one or more LSD1 inhibitors by, or by about, 1%, 5%, 10%, 15%, 20%, 25%, 30%, 35%, 40%, 45%, 50%, 55%, 60%, 65%, 70%, 75%, 80%, 85%, 90%, 95%, or a range between any two of these values. The sensitization of the cancer cells can increase the responsiveness of the subject having the cancer cells to the one or more LSD1 inhibitors by at least, or by at least about, 1%, 5%, 10%, 15%, 20%, 25%, 30%, 35%, 40%, 45%, 50%, 55%, 60%, 65%, 70%, 75%, 80%, 85%, 90%, 95%, or a range between any two of these values. The increase of the responsiveness of the subject having the cancer cells is, in some embodiments, relative to the subjects untreated with the composition.

The method can comprise determining sensitization of the cancer cells to the one or more LSD1 inhibitors after being contacted with the PLK1 inhibitor. The method can comprise contacting the cancer cells with the one or more LSD1 inhibitors concurrently and/or after being contacted with the PLK1 inhibitor. In some embodiments, contacting the cancer cells with the one or more LSD1 inhibitors occurs in the body of a subject. The subject can be a mammal, for example human. The subject can be, for example, a subject that did not respond to, or is known to be resistant to, LSD1 inhibitors alone. The subject can be, for example, a subject that had prior treatment with one of the one or more LSD1 inhibitors. In some embodiments, the method comprises determining the response of the subject to the one or more LSD1 inhibitors.

### EXAMPLES

Some aspects of the embodiments discussed above are disclosed in further detail in the following example, which are not in any way intended to limit the scope of the present disclosure.

### Example 1

### Onvansertib Synergizes with ORY-1001 in SCLC Cell Lines

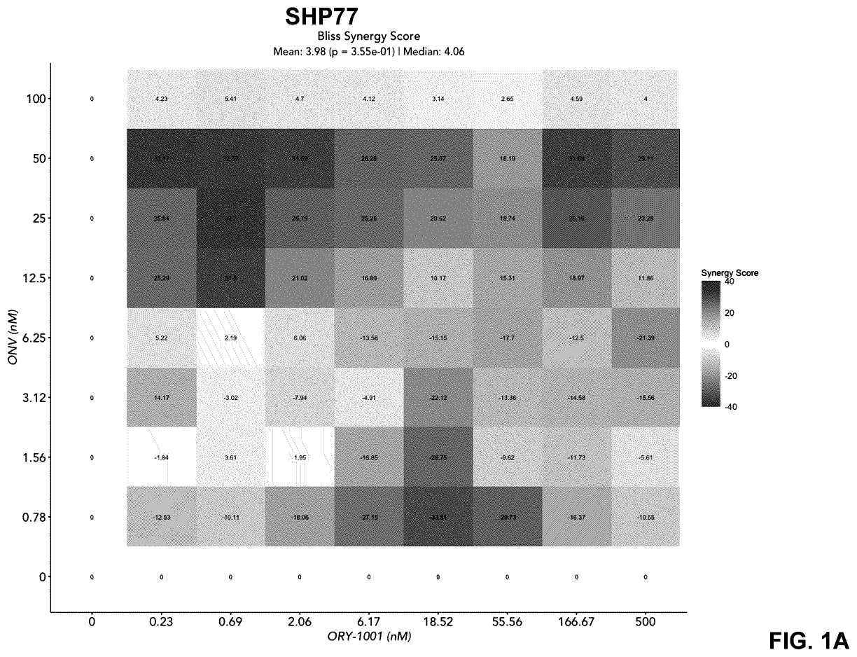

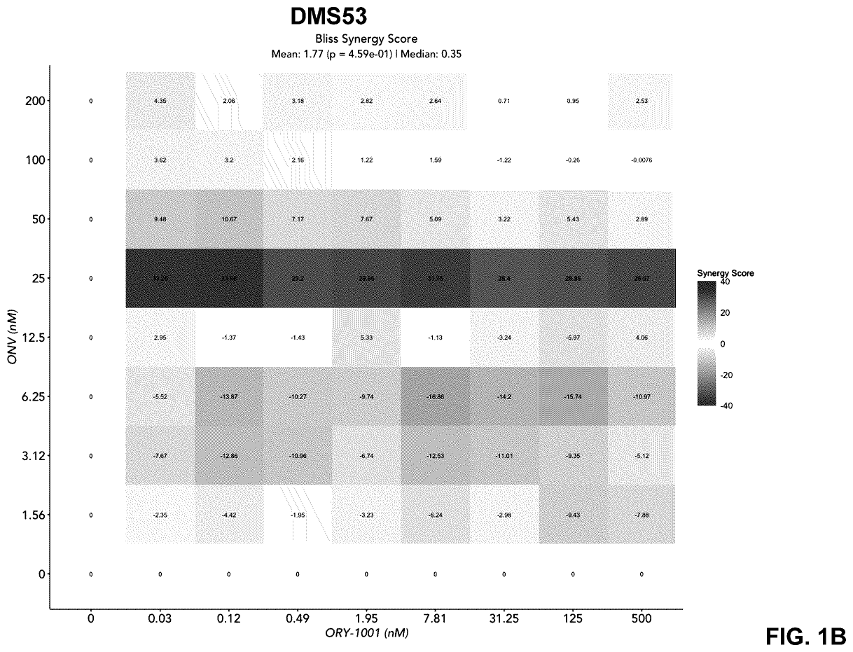

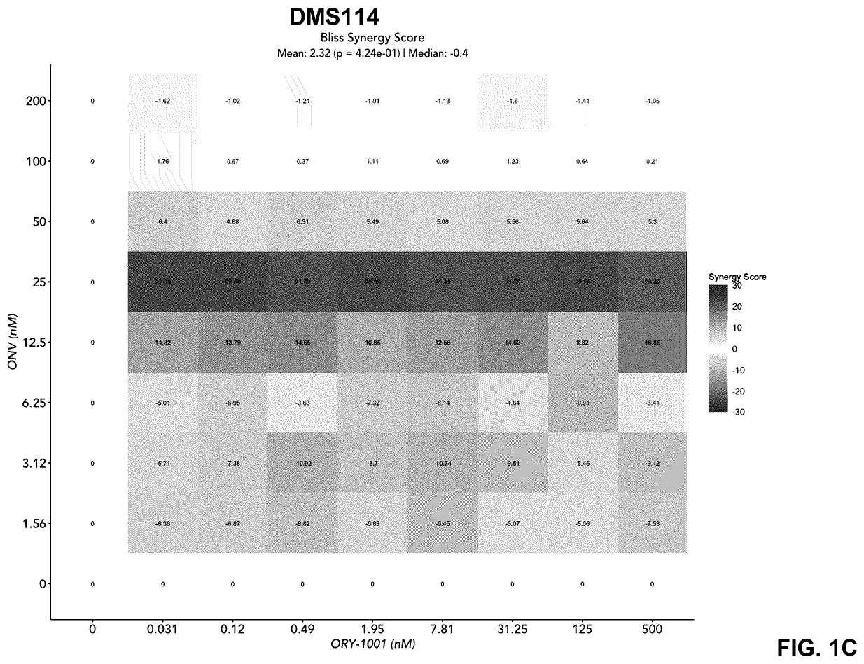

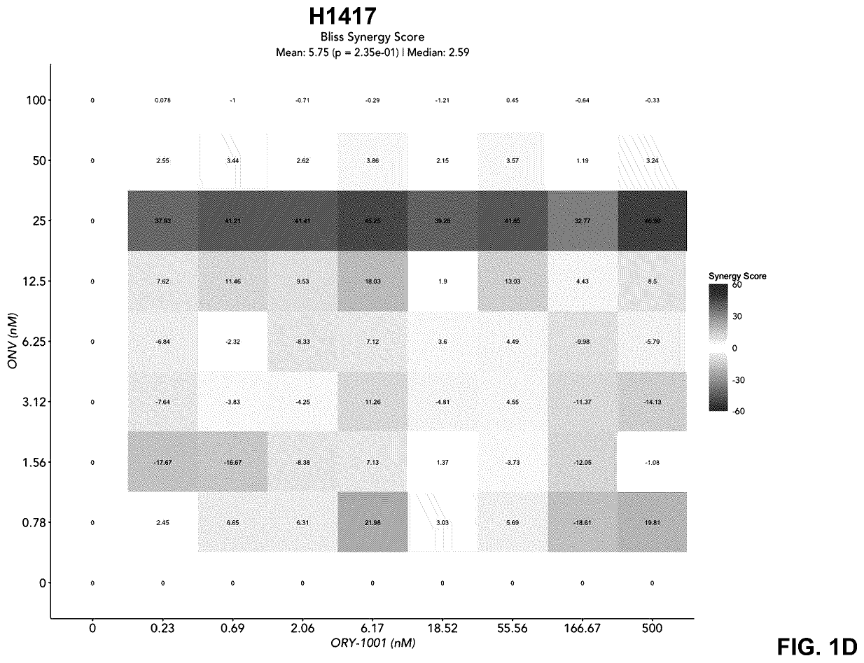

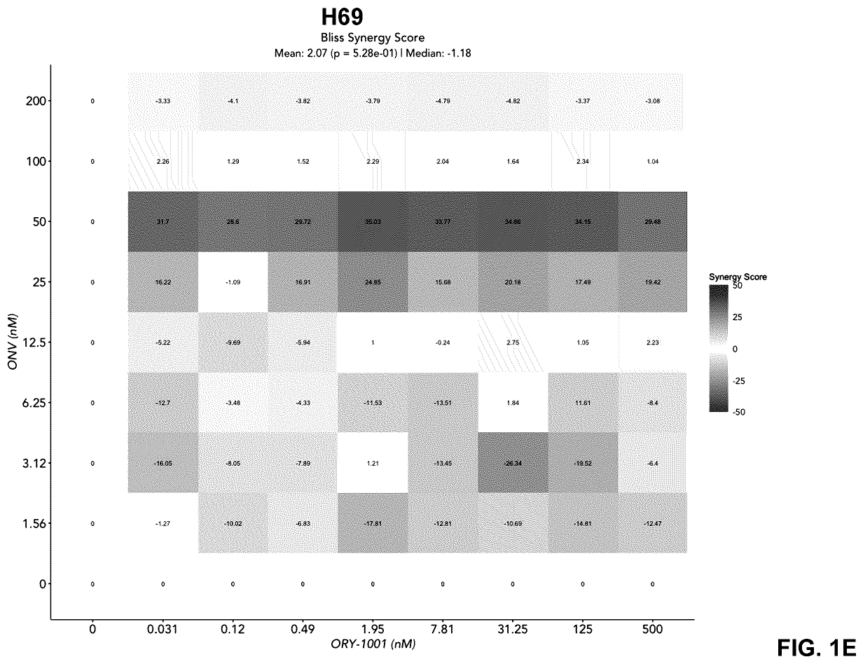

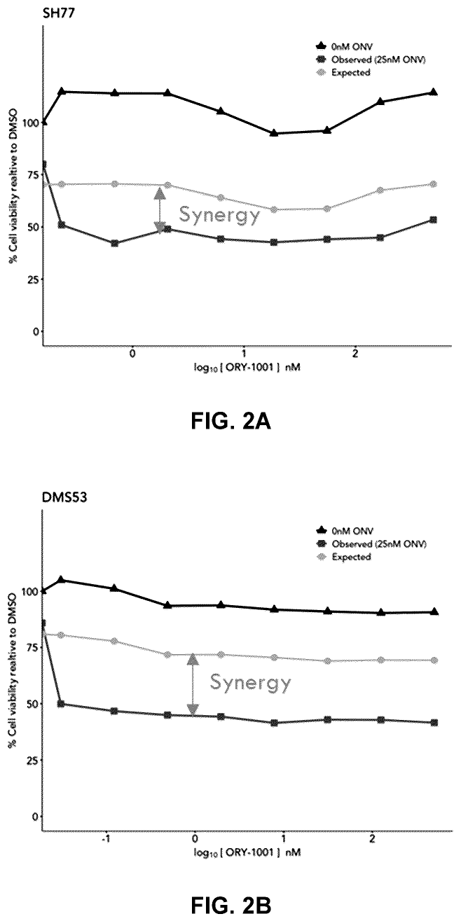

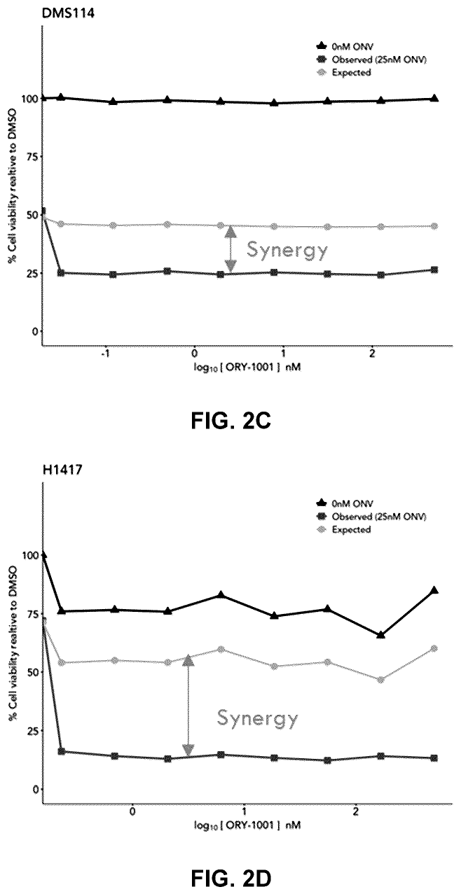

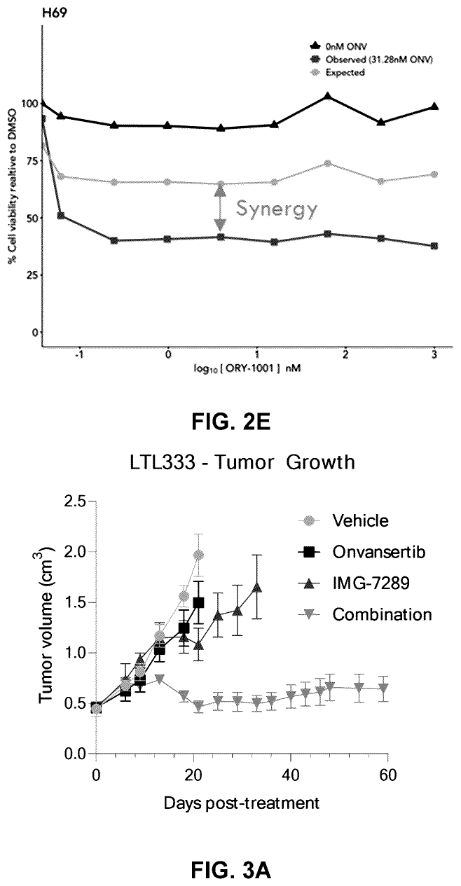

Five small cell lung cancer (SCLC) cell lines were treated with various doses of onvansertib and ORY-1001 for 6 days. Cell viability was measured using CellTiterGlo® assays. Synergy of the drug combination was calculated using SynergyFinder application (Ianevski et al., Nucleic Acids Research, 2020, 48 (W1), W488-W493). The Bliss independence synergy scores for each cell line are shown in FIGS. 1A-1E (results from SHP77, DMS53, DMS114, H1417, and H69 cell lines, respectively), in which positive scores indicate synergy between onvansertib and ORY-1001 at given concentrations. In these cell lines, at ovansertib concentration of about 25 nM, strong synergistic efficacy (as indicated by high positive synergy scores) was observed over a wide range of ORY-1001 concentrations (about 0.03-500 nM). Further, viability in cells treated with various doses of ORY-1001 in the absence or presence of onvansertib are shown in FIGS. 2A-2E (results from SHP77, DMS53, DMS114, H1417, and H69 cell lines, respectively). Expected viability was calculated using the Bliss independence model of drug additivity. The area between the expected viability and the observed viability in the presence of onvansertib (about 25 nM) indicates synergy between onvansertib and ORY-1001. For example, in DMS53 cells (FIG. 2B), the expected and observed viabilities for combinations of onvansertib and ORY-1001 are about 75% and about 50%, respectively (i.e. 25% expected vs. 50% observed cancer cell inhibition), which represents an about 100% increase in efficacy with the drug combination compared to the combined inhibition caused by ORY-1001 alone plus onvansertib alone. Collectively, these results demonstrated an enhanced or synergistic efficacy in inhibiting cancer progression using a combination of ORY-1001 and onvansertib.

### Example 2

### Antitumor Activity of Onvansertib and IMG-7289 in Prostate Cancer Neuroendocrine PDX Models

In this example, the efficacies of onvansertib alone and in combination with IMG-7289 were evaluated in a prostate cancer neuroendocrine patient-derived xenograft (PDX) model referred to as LTL331R78 model. The LTL331R78 model expresses the neuroendocrine transcription factor ASCL1. This model is also characterized by the presence of TP53 mutation and the loss of RB1 and PTEN expression.

Mice were transplanted with tumor fragment and randomized to receive treatment. Mice (5 mice/group) were then treated 5 days a week for 59 days with the following: (1) vehicle; (2) Onvansertib (60 mg/kg); (3) IMG-7289 (40 mg/kg); or (4) the combination of Onvansertib (60 mg/kg) and IMG-7289 (40 mg/kg). Tumor volume was measured.

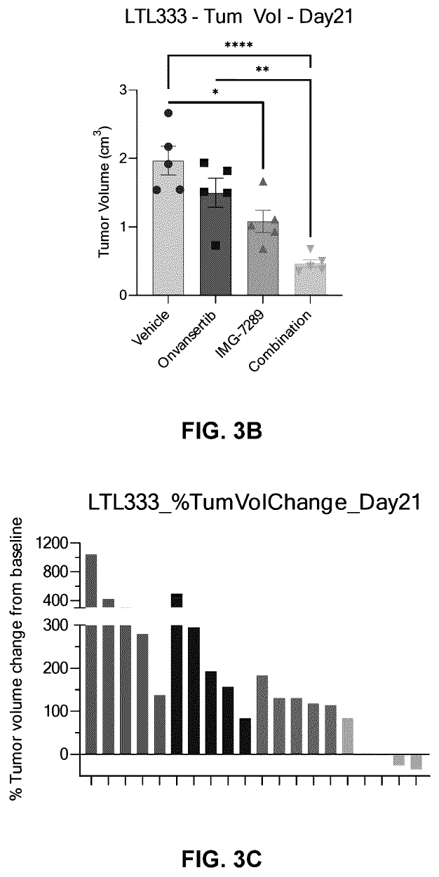

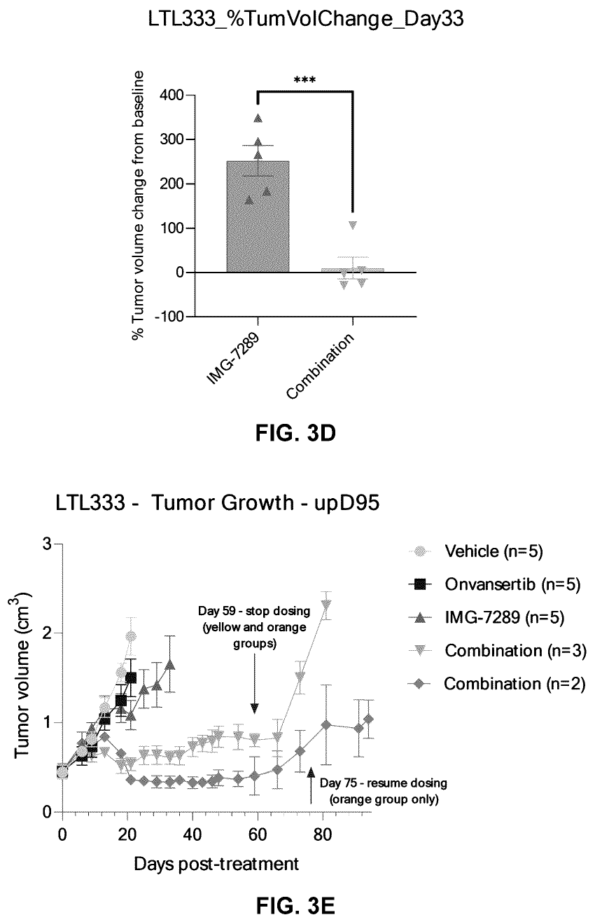

FIG. 3A is a plot showing tumor volume in the LTL331R78 model treated with vehicle, onvansertib, IMG-7289, or onvansertib and IMG-7289 combination. The combination of onvansertib and IMG-7289 showed greater and more durable tumor growth inhibition in comparison with vehicle, onvansertib or IMG-7289 alone.

FIG. 3B is a plot showing tumor volume in the LTL331R78 model at Day 21. At Day 21, IMG-7286 alone or in combination with onvansertib induced significant tumor growth inhibition compared to vehicle (45% and 76%, respectively). One-way ANOVA with multiple comparisons was used to test statistical differences at Day 21, *p<0.05, **p<0.01, ****p<0.0001. FIG. 3C is a plot showing the percentage of tumor volume change from baseline in the LTL331R78 model at Day 21. Tumor regression was also observed in 3 of the 5 mice in the combination group, while none in the other groups. FIG. 3D is a plot showing the percentage of tumor volume change from baseline at Day 33. At Day 33, the anti-tumor activity in the combination group was greater compared to the IMG-7286 monotherapy group. Unpaired t-test was used to test statistical differences at Day 33, ***p<0.001.

Treatment was resumed on Day 75 for 2 of the 5 mice in the combination group. FIG. 3E shows tumor volume in the LTL331R78 model with the resumed treatment in the combination group for up to 95 days. The data indicates that resuming treatment with the combination of onvansertib and IMG-7286 slowed down tumor progression, thus suggesting that the combination can be efficacious after 15 days of drug holiday.

### Example 3

### Antitumor Activity of Onvansertib and IMG-7289 in Neuroendocrine PDX Models

In this example, the efficacies of onvansertib alone and in combination with IMG-7289 were evaluated in an EF1 neuroendocrine PDX model. The EF1 model expresses the neuroendocrine transcription factor NeuroD1.

Two sets of mice were transplanted with tumor fragment and randomized to receive treatment. Mice (4-8 mice/group) were then treated 5 days a week for up to 32 days with the following: (1) vehicle; (2) Onvansertib (60 mg/kg); (3) IMG-7289 (40 mg/kg); or (4) the combination of Onvansertib (60 mg/kg) and IMG-7289 (40 mg/kg). Tumor volume was measured. One-way ANOVA with multiple comparisons was used to test statistical differences, *p<0.05, **p<0.01.

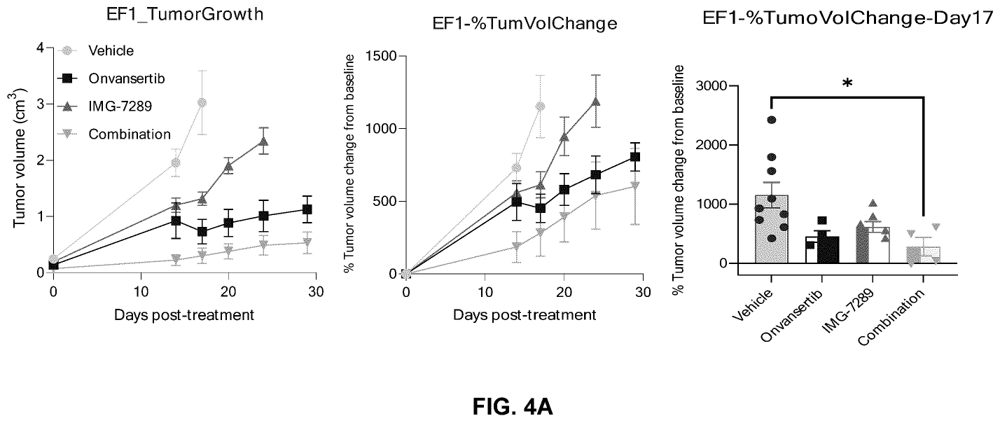

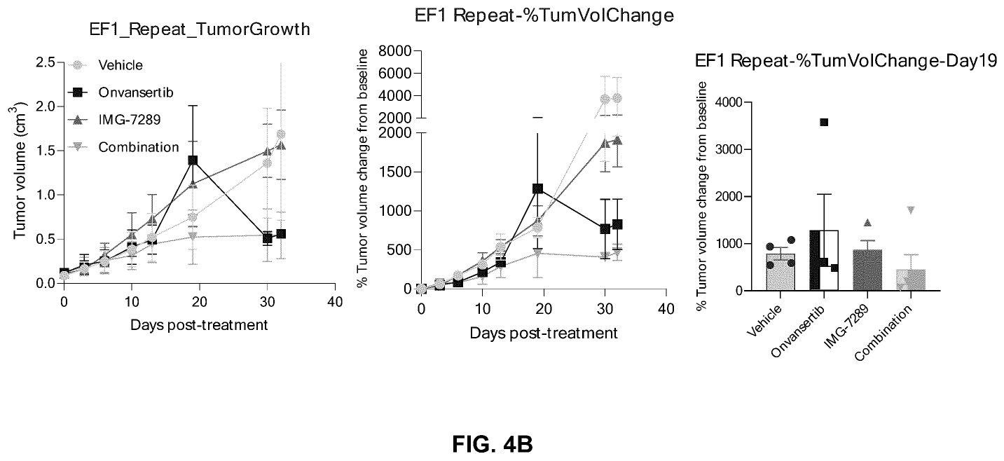

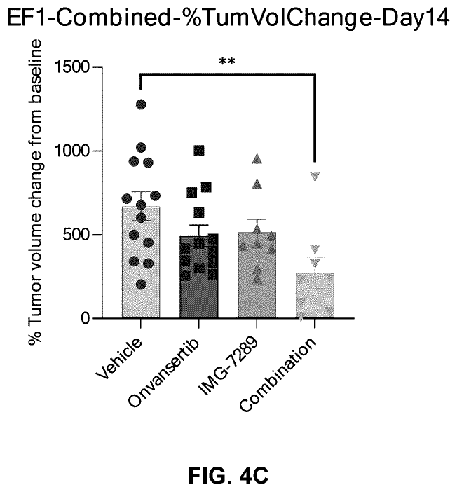

FIG. 4A provides plots showing changes in tumor volume (left panel) and the percentage of tumor volume change from baseline (middle panel) in the EF1 model treated with vehicle, onvansertib, IMG-7289, or onvansertib and IMG-7289 combination in a first set of mice. The right panel in FIG. 4A is a plot showing the percentage of tumor volume change from baseline at Day 17. FIG. 4B provides plots showing changes in tumor volume (left panel) and the percentage of tumor volume change from baseline (middle panel) in the treated EF1 model in a second set of mice, as well as the percentage of tumor volume change from baseline at Day 17 (right panel). In both experiments, the combination of onvansertib and IMG-7289 induced a decrease in tumor growth in comparison to the control.

FIG. 4C is a plot showing the percentage of tumor volume change from baseline at Day 14. The combination of onvansertib and IMG-7289 induced a significant tumor growth inhibition (about 75%) and a decrease in the percentage of tumor volume change (about 60%) compared to the control, while no significant difference was observed in the monotherapy groups.

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. As used in this specification and the appended claims, the singular forms “a,” “an,” and “the” include plural references unless the context clearly dictates otherwise. Any reference to “or” herein is intended to encompass “and/or” unless otherwise stated.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A method of treating cancer, the method comprising: administrating a lysine-specific histone demethylase 1 (LSD1) inhibitor and a Polo-like kinase 1 (PLK1) inhibitor to a subject with cancer, thereby inhibiting or reducing progression of the cancer in the subject.

2. The method of claim 1, wherein the subject has head and neck cancer, lung cancer, intrahepatic cholangiocarcinoma (iCCA), gastric cancer, urothelial cancer, endometrial cancer, cervical cancer, rhabdomyosarcoma, cholangiocarcinoma, glioblastoma, low-grade glioma, ovarian cancer, prostate adenocarcinoma, thyroid carcinoma, endometrial cancer, gallbladder cancer, breast cancer, pancreatic ductal adenocarcinoma, prostate cancer, sarcoma, or a combination thereof; and optionally the prostate cancer is neuroendocrine prostate cancer.

3. The method of any one of claims 1-2, comprising identifying a subject with cancer as a subject having LSD1 gene amplification, high LSD1 expression, or LSD1 overexpression.

4. The method of any one of claims 1-3, wherein the subject has LSD1 gene amplification, high LSD1 expression, or LSD1 overexpression.

5. The method of any one of claims 1-4, wherein the subject has prostate cancer, sarcoma, lung adenocarcinoma, breast cancer, hepatocellular carcinoma, esophageal cancer, lung cancer, neuroblastoma, acute myeloid leukemia, or a combination thereof.

6. The method of any one of claims 1-5, wherein the PLK1 inhibitor and the LSD1 inhibitor are co-administered simultaneously, or are administered sequentially.

7. The method of any one of claims 1-6, wherein the administration of the PLK1 inhibitor and/or the LSD1 inhibitor is oral administration.

8. The method of any one of claims 1-7, wherein the inhibition of cancer progression is greater than the combined inhibition of progression caused by the LSD1 inhibitor alone plus the PLK1 inhibitor alone.

9. The method of any one of claims 1-8, wherein the subject achieves a complete response.

10. The method of any one of claims 1-9, wherein the subject has received a prior LSD1 inhibitor treatment.

11. The method of any one of claims 1-10, wherein the subject did not respond to treatment with the LSD1 inhibitor alone or is known to be resistant to an LSD1 inhibitor therapy.

12. The method of any one of claims 1-11, wherein the LSD1 inhibitor and the PLK1 inhibitor are each administered to the subject in a cycle of at least twice or at least give times within a week.

13. The method of any one of claims 1-12, wherein the LSD1 inhibitor, the PLK1 inhibitor, or both are administered in a cycle of at least 7 days, and optionally wherein each cycle of treatment is at least about 21 days, and further optionally from about 14 days to about 28 days.

14. The method of any one of claims 1-13, wherein the PLK1 inhibitor is administered on at least four days in the cycle.

15. The method of any one of claims 1-14, wherein the PLK1 inhibitor is not administered on at least one day in the cycle.

16. The method of any one of claims 1-15, wherein the LSD1 inhibitor is administered once daily or twice daily.

17. The method of any one of claims 1-16, wherein the LSD1 inhibitor is administered once daily.

18. The method of claims 1-17, wherein the subject undergoes at least two cycles of the administration of the LSD1 inhibitor and the PLK1 inhibitor.

19. The method of any one of claim 1-18, wherein the LSD1 inhibitor is a reversible LSD1 inhibitor.

20. The method of any one of claims 1-18, wherein the LSD1 inhibitor is tranylcypromine and derivatives thereof, bizine, RN-1 (hydrochloride), GSK LSD1 dihydrochloride, OG-L002, trans-N-((2,3-dihydrobenzo[b](1,4]dioxin-6-yl)methyl)-2-phenylcyclopropan-1-amine, trans-N-((2-methoxypyridin-3-yl)methyl)-2-phenylcyclopropan-1-amine, ORY-1001, OG86, GSK2879552, IMG-7289, INCB059872, CC-90011, ORY-2001, MC2580, DDP38003, (R)-4-[5-(Pyrrolidin-3-ylmethoxy)-2-p-tolyl-pyridin-3-yl]-benzonitrile, 1-(4-methyl-1-piperazinyl)-2-[[(1R*,2S*)-2-[4-phenylmethoxy)phenyl]cyclopropyl]amino]ethanone, N-[4-[trans-2-aminocyclopropyl]phenyl]-4-(4-methylpiperazin-1-yl)benzamide, namoline, pargyline, SP2509, S1201, S2101, C76, GSK690, Cpd 2d, RN7, RO7051790, SYHA1807, TAS1440, SP-2577 (seclidemstat), 2-PFPA, NCL-1, HCI-2509, or a pharmaceutically acceptable salt, hydrate, solvate or prodrug thereof.

21. The method of any one of claims 1-20, wherein a maximum concentration (Cₘₐₓ) of onvansertib in a blood of the subject is from about 1 pg/mL to about 10 μg/mL.

22. The method of any one of claims 1-21, wherein an area under curve (AUC) of a plot of a concentration of onvansertib in a blood of the subject over time is from about 1 pg·h/mL, to about 100 μg·h/mL.

23. The method of any one of claims 1-22, wherein a time to reach a maximum concentration (Tₘₐₓ) of onvansertib in a blood of the subject is from about 1 hour to about 24 hours.

24. The method of any one of claims 1-23, wherein an elimination half-life (T_{1/2}) of onvansertib in a blood of the subject is from about 10 hours to about 100 hours

25. The method of any one of claims 1-24, wherein the PLK1 inhibitor is a dihydropteridinone, a pyridopyrimidine, a aminopyrimidine, a substituted thiazolidinone, a pteridine derivative, a dihydroimidazo[1,5-f]pteridine, a metasubstituted thiazolidinone, a benzyl styryl sulfone analogue, a stilbene derivative, or any combination thereof.

26. The method of any one of claims 1-24, wherein the PLK1 inhibitor is onvansertib, BI2536, Volasertib (BI 6727), GSK461364, AZD1775, CYC140, HMN-176, HMN-214, rigosertib (ON-01910), MLN0905, TKM-080301, TAK-960 or Ro3280.

27. The method of any one of claims 1-24, wherein the PLK1 inhibitor is onvansertib, and optionally wherein onvansertib is administered at a dose in the range of 6 mg/m²-90 mg/m².

28. The method of claim 27, wherein a maximum concentration (Cₘₐₓ) of onvansertib in a blood of the subject is from about 100 nmol/L to about 1500 nmol/L.

29. The method of any one of claims 27-28, wherein an area under curve (AUC) of a plot of a concentration of onvansertib in a blood of the subject over time is from about 1000 nmol/L·hour to about 400000 nmol/L·hour.

30. The method of any one of claims 27-29, wherein a time to reach a maximum concentration (Tₘₐₓ) of onvansertib in a blood of the subject is from about 1 hour to about 5 hours.

31. The method of any one of claims 27-30, wherein an elimination half-life (T_{1/2}) of onvansertib in a blood of the subject is from about 10 hours to about 60 hours.

32. The method of any one of claims 1-31, wherein the subject has received at least one prior cancer treatment, and optionally where the prior treatment does not comprise the use of a LSD1 inhibitor, a PLK inhibitor, or both; and further optionally the PLK inhibitor is onvansertib.

33. The method of any one of claims 1-32, wherein the subject was in remission for cancer, optionally wherein the subject in remission for cancer was in complete remission (CR) or in partial remission (PR).

34. The method of any one of claims 1-33, further comprising determining cancer status of the subject.

35. The method of any one of claims 1-34, further comprising determining responsiveness of the subject to the treatment of LSD1 inhibitor and PLK1 inhibitor.

36. The method of any one of claims 1-35, further comprising administering one or more cancer therapeutics or therapies for the cancer.

37. The method of any one of claims 1-36, the subject is human.

38. A method of sensitizing cancer cells to a lysine-specific histone demethylase 1 (LSD1) inhibitor, the method comprising: contacting cancer cells with a composition comprising a Polo-like kinase 1 (PLK1) inhibitor, thereby sensitizing the cancer cells to the LSD1 inhibitor.

39. The method of claim 38, wherein the PLK1 inhibitor is onvansertib.

40. The method of any one of claims 38-39, wherein contacting cancer cells with the composition occurs in vitro, ex vivo, and/or in vivo.

41. The method of any one of claims 38-40, wherein contacting cancer cells with the composition is in a subject, and optionally wherein the subject did not respond to, or is known to be resistant to, the LSD1 inhibitor.

42. The method of claim 41, wherein the subject had prior treatment with the LSD1 inhibitor.

43. The method of any one of claims 41-42, wherein the subject is a mammal, and optionally the mammal is human.

44. The method of any one of claims 38-43, comprising determining sensitization of the cancer cells to the LSD1 inhibitor after being contacted with the composition.

45. The method of any one of claims 38-44, comprising contacting the cancer cells with the LSD1 inhibitor, and optionally wherein contacting the cancer cells with the LSD1 inhibitor occurs in the subject.

46. The method of claim 45, comprising determining the response of the subject to the LSD1 inhibitor.

47. The method of any one of claims 45-46, wherein contacting the cancer cells with the LSD1 inhibitor is concurrent with the contacting the cancer cells with the composition, or after the contacting the cancer cells with the composition.

48. The method of any one of claims 38-47, wherein the cancer cells are cells comprising LSD1 gene amplification, and/or wherein the cancer cells are cells having high LSD1 expression or LSD1 overexpression.

49. A kit, comprising:
a Polo-like kinase 1 (PLK1) inhibitor; and
a manual providing instructions for co-administrating the PLK1 inhibitor with a lysine-specific histone demethylase 1 (LSD1) inhibitor to a subject in need thereof for treating cancer.

50. The kit of claim 49, wherein the cancer is head and neck cancer, lung cancer, intrahepatic cholangiocarcinoma (iCCA), gastric cancer, urothelial cancer, endometrial cancer, cervical cancer, rhabdomyosarcoma, cholangiocarcinoma, glioblastoma, low-grade glioma, ovarian cancer, prostate adenocarcinoma, thyroid carcinoma, endometrial cancer, gallbladder cancer, breast cancer, pancreatic ductal adenocarcinoma, prostate cancer, sarcoma, or a combination thereof, and optionally the prostate cancer is neuroendocrine prostate cancer.

51. The kit of claim 50, wherein the cancer is prostate cancer, sarcoma, lung adenocarcinoma, breast cancer, hepatocellular carcinoma, esophageal cancer, lung cancer, neuroblastoma, acute myeloid leukemia, or a combination thereof.

52. The kit of any one of claims 49-51, wherein the subject has LSD1 gene amplification, high LSD1 expression, LSD1 overexpression, or a combination thereof.

53. The kit of any one of claims 49-52, wherein the PLK1 inhibitor is onvansertib.

54. The kit of any one of claims 49-53, wherein the instructions comprise instructions for co-administrating the PLK1 inhibitor and the LSD1 inhibitor simultaneously or for administering the PLK1 inhibitor and the LSD1 inhibitor sequentially.

55. The kit of any one of claims 49-54, wherein the instructions comprise instructions for administering of the PLK1 inhibitor and/or the LSD1 inhibitor orally.

56. The kit of any one of claims 49-55, wherein the instructions comprise instructions that the subject has received a prior LSD1 inhibitor treatment or the subject did not respond to treatment with the LSD1 inhibitor alone.

57. The kit of any one of claims 49-56, wherein the instructions comprise instructions the subject is known to be resistant to an LSD1 inhibitor therapy.

58. The kit of any one of claims 53-57, wherein the instructions comprise instructions for administering each of the LSD1 inhibitor and onvansertib to the subject in a cycle of at least twice or at least five times within a week.

59. The kit of any one of claims 53-58, wherein the instructions comprise instructions for administering the LSD1 inhibitor, onvansertib, or both are in a cycle of at least 7 days or at least about 21 days; and optionally each cycle of treatment is from about 14 days to about 28 days.

60. The kit of any one of claims 59-59, wherein the instructions comprise instructions for administering onvansertib on at least four days in the cycle.

61. The kit of any one of claims 59-60, wherein the instructions comprise instructions for not administering onvansertib on at least one day in the cycle.

62. The kit of any one of claims 49-61, wherein the instructions comprise instructions for administrating the LSD1 inhibitor daily, and optionally once daily or twice daily.

63. The kit of any one of claims 53-62, wherein the instructions comprise instructions for administrating the LSD1 inhibitor and onvansertib for at least two cycles.

64. The kit of any one of claims 49-63 wherein the LSD1 inhibitor is a reversible LSD1 inhibitor.

65. The kit of any one of claims 49-64, wherein the LSD1 inhibitor is tranylcypromine and derivatives thereof, bizine, RN-1 (hydrochloride), GSK LSD1 dihydrochloride, OG-L002, trans-N-((2,3-dihydrobenzo[b](1,4]dioxin-6-yl)methyl)-2-phenylcyclopropan-1-amine, trans-N-((2-methoxypyridin-3-yl)methyl)-2-phenylcyclopropan-1-amine, ORY-1001, OG86, GSK2879552, IMG-7289, INCB059872, CC-90011, ORY-2001, MC2580, DDP38003, (R)-4-[5-(Pyrrolidin-3-ylmethoxy)-2-p-tolyl-pyridin-3-yl]-benzonitrile, 1-(4-methyl-1-piperazinyl)-2-[[(1R*,2S*)-2-[4-phenylmethoxy)phenyl]cyclopropyl]amino]ethanone, N-[4-[trans-2-aminocyclopropyl]phenyl]-4-(4-methylpiperazin-1-yl)benzamide, namoline, pargyline, SP2509, S1201, S2101, C76, GSK690, Cpd 2d, RN7, RO7051790, SYHA1807, TAS1440, SP-2577 (seclidemstat), 2-PFPA, NCL-1, HCI-2509, or a pharmaceutically acceptable salt, hydrate, solvate or prodrug thereof.

66. The kit of any one of claims 53-65, wherein the instructions comprise instructions for administering onvansertib at a dose in the range of 6 mg/m²-90 mg/m².

67. The kit of any one of claims 49-66, wherein the subject has received at least one prior treatment for the cancer.

68. The kit of claim 67, where the prior treatment does not comprise the use of a LSD1 inhibitor, onvansertib, or both.

69. The kit of any one of claims 49-68, wherein the subject was in remission for cancer.

70. The kit of claim 69, wherein the subject in remission for cancer was in complete remission (CR) or in partial remission (PR).

71. The kit of any one of claims 49-70, further comprising the LSD1 inhibitor.
